(19) United States
(12) Patent Application Publication
(10) Pub. No.: US 2026/0248951 A1
(43) Pub. Date: Aug. 27, 2026

(54) CIRCULATING TUMOR CELLS (CTC) CAPTURE APPARATUS AND METHODS

(71) Applicant: ONCONQUER MEDICAL LTD, Bustan Hagalil (IL)

(72) Inventors: Moshe SHOHAM, Hoshaya (IL); Tze Chuen NG, Hong Kong (CN); Or SAMOOCHA, Bustan Hagalil (IL); Gadi SICHERMAN, Hoshaya (IL); Igal RASIN, Haifa (IL)

(21) Appl. No.: 19/157,042

(22) PCT Filed: Feb. 16, 2024
(86) PCT No.: PCT/IB2024/051474

§ 371 Date: Aug. 15, 2025

Related U.S. Application Data
(60) Provisional application No. 63/576,533, filed on Feb. 16, 2023.
(60) Provisional application No. 63/498,219, filed on Apr. 25, 2023.

(51) Int. Cl. A61K 47/69; A61B 34/00; A61F 2/01; A61K 47/68; A61N 5/06

(52) U.S. Cl. CPC A61K 47/6923; A61B 34/73; A61F 2/01; A61K 47/6851; A61N 5/0603; A61B 2034/731; A61F 2002/016; A61F 2210/0004; A61F 2210/009; A61F 2250/0067; A61N 2005/0602; A61N 2005/063; A61N 2005/0661

(57) ABSTRACT

Apparatus and methods are described for capturing circulating tumor cells (CTC) in vivo. An intravascular implantable implant body (20, 54, 84) is implanted in a blood vessel of a subject. Antibody-conjugated magnetic particles (80) are configured, upon being released into a bloodstream of the subject, to (a) magnetically attach to the implant body (20, 54, 84), and (b) selectively bind circulating tumor cells (CTC) in the blood vessel. A releasing element (82, 85) periodically releases antibody-conjugated magnetic particles (80) into the subject's bloodstream such as to replenish the implant body (20, 54, 84) with antibody-conjugated magnetic particles (80). Other applications are also described.

### CROSS-REFERENCES TO RELATED APPLICATIONS

The present application claims priority from U.S. Provisional Patent Application No. 63/576,533 to Shoham et al., filed Feb. 16, 2023, entitled “Halting of circulating tumor cells in cancer patients”, and from U.S. Provisional Patent Application No. 63/498,219 to Shoham et al., et al., filed Apr. 25, 2023, entitled “Intravascular device and method for denaturing circulating tumor cells”, both of which are incorporated herein by reference.

### FIELD OF APPLICATIONS OF THE INVENTION

Some applications of the present invention generally relate to devices and methods for capturing and inactivating circulating tumor cells (CTC). More specifically, some applications of the present invention relate to implantable apparatus and techniques for diagnosing and removing CTC from the blood circulation.

### BACKGROUND

Circulating tumor cells (CTC) originate from one or more primary tumors that have shed tumor cells into the bloodstream. Cancer metastasis may occur if these CTC act as seeds for the growth of additional secondary tumors at locations remote from the original primary tumor. CTC are generally rare in the blood stream. Approximately 1-100 cells are found in 10⁶-10⁸ red blood cells per milliliter of blood, with less than 0.01% of CTC that metastasize. Circulating tumor cells (CTC) are believed to be involved in the development of metastasis in several types of cancer, including colorectal, prostate and breast cancer. There is evidence that CTC in blood circulation may be associated with a poor prognosis for certain types of cancer. For example, in breast cancer, the presence of CTC has been linked to an increased risk of distant metastasis and a lower overall survival. Similarly, CTC detection in prostate cancer subjects has been linked with a higher risk of disease progression and a poor diagnosis. Additionally, the life expectancy of cancer subjects is correlated with the number of CTC in the bloodstream.

As metastatic cancer is associated with low survival rates, the eradication of CTC may help to prevent the growth of secondary tumors in a subject's body. Additionally, the detection and analysis of CTC may assist early subject prognosis and determine appropriate tailored treatments. Researchers and clinicians are interested in the detection and analysis of CTC because it may shed light on the biology of cancer progression and influence the choice of treatment options for specific subjects.

### SUMMARY

In accordance with some applications of the present invention, an apparatus comprising an in-situ implant device, for selectively capturing circulating tumor cells (CTC) from the circulatory system of a subject, is provided. Typically, the implant is deployed in a blood vessel of the subject to capture CTC in vivo and remove the CTC from the bloodstream. Typically, capturing the CTC on the implant results in inactivation and destruction of the CTC in vivo. Typically, the CTC are passively destroyed by being captured by the implant and exposed to the environment in the blood vessel. Additionally, or alternatively, For some the CTC are actively denatured following capturing by the implant (as will be described hereinbelow). Typically, halting the CTC reduces incubation and formation of metastasis by stopping traveling of the CTC through the bloodstream. For some applications, the apparatus provided in accordance with some applications of the present invention, target the circulating tumor cells (CTC) in the bloodstream for diagnostic purposes, e.g., to monitor disease progression and/or tailor a treatment plan for the subject.

In accordance with some applications of the present invention, the implantable CTC-capturing apparatus that is deployed in the subject's blood vessel is used with antibodies that selectively bind to the CTC thereby capturing the CTC. Capturing of the CTC by the antibodies typically results in inactivation of the CTC by causing death of the CTC, e.g., due to shear force applied by the blood stream and/or lack of nutrients that are necessary for survival of the CTC, and/or through the action of the body's immune system. It is hypothesized by the inventors of the present application that the CTC are rendered non-viable within several hours of binding to the antibodies. In such a manner the risk of metastasis formation in cancerous diseases can be reduced.

For some applications, the implant is a stationary implant (such as a stent-like structure or a multiple wire implant positioned in a stationary manner in the blood vessel) that is infused with antibodies that selectively bind the CTC. For some applications, the antibodies are immobilized onto the implant structure. For some such applications, binding of the CTC to the antibodies is caused by impact of the CTC with the antibodies in the implant. Impact attachment of the CTC to the antibodies is caused when CTC moving through the bloodstream pass by/through the stationary implant positioned in the vasculature of the subject and collide with the antibodies in the implant.

For some applications, the implant comprises a magnetic implant and the antibodies contain a magnetic component that upon pairing with the CTC is attracted to the magnetic implant, which is held stationary in the subject's vasculature, thereby removing the CTC from the bloodstream and reducing the likelihood of metastasis. For some such applications, the apparatus and techniques provided herein allow binding of CTC to the antibodies while allowing flow of the antibodies in the blood vessel while confining the antibodies to a defined area within a blood vessel, and preventing uncontrolled, systemic and continuous circulation of the antibodies throughout the bloodstream.

For some applications in which the implant and the antibodies comprise a magnetic component, the antibodies are manipulated by application of a magnetic field such that the antibodies are released from the implant by a magnetic force and allowed to float freely within a defined area of the blood vessel, generally in the vicinity of the implant, so as to capture CTC within the bloodstream in the defined area. The antibodies are re-attracted to the implant by an alternate magnetic force such that the antibodies return and remain in the implant and are not allowed to circulate freely in the blood circulation.

Generally, antibodies have a half-life of several days, thereby requiring replenishing in order for the implant to be effective against CTC over a desired period of time (e.g., weeks). In accordance with some applications of the present invention, replenishing of the antibodies is done by intravenous (IV) injection of antibodies directly into the bloodstream. Typically, the magnetic component in the antibodies is attracted to the magnetic component in the implant, such that the injected antibodies are drawn to the implant and do not circulate uncontrollably in the bloodstream. Alternatively, or additionally, for some applications, the implant comprises an at least partially biodegradable scaffold comprising antibodies, which exposes and releases viable antibodies into the body as the implant biodegrades.

In accordance with additional applications of the present invention, devices, systems, and methods for capturing CTC and exposing them to ultraviolet light for an effective period of time to denature the cells in situ so that they are unable to circulate metastasize at another site in the subject's body are provided. For some such applications, an implant device comprising a central magnetic, flexible core, surrounded by side-emitting UV optical fibers is implanted in a blood vessel of the subject (e.g., by being subcutaneously injected into the blood vessel) and remains in place for days to weeks. The optical fibers can be coated with an anti-coagulant. Magnetic particles (e.g., containing a ferromagnetic material) are conjugated to one or more biomarkers (e.g., antibodies for selectively binding CTC). The magnetic particles remain in contact with the implant via magnetic forces while the implant device is deployed in the blood vessel. The implant device is attachable to a UV generator. In use, CTC in the blood circulation attach to the antibodies on the implant device, and UV waves are periodically delivered to denature captured CTC.

Although some applications of the present invention are described with respect to antibodies or other biomarkers that are configured to bind with circulating tumor cells, the scope of the present disclosure includes applying the apparatus and method described herein to the removal of microorganisms (such as bacteria or viruses) from the bloodstream, by utilizing antibodies that are configured to bind with such microorganisms, mutatis mutandis.

There is therefore provided, in accordance with some embodiments of the present invention, apparatus for capturing circulating tumor cells (CTC) in vivo, and for use with an intravascular implantable implant body that is implanted in a blood vessel of a subject, the apparatus including:
antibody-conjugated magnetic particles configured, upon being released into a bloodstream of the subject, to (a) magnetically attach to the implant body, and (b) selectively bind circulating tumor cells (CTC) in the blood vessel, and
a releasing element that is configured to periodically release antibody-conjugated magnetic particles into the subject's bloodstream such as to replenish the implant body with antibody-conjugated magnetic particles.

In some embodiments, the releasing element includes a biodegradable material that is degradable into the blood vessel, and the antibody-conjugated magnetic particles are embedded in the biodegradable material, and are configured to become released into the blood vessel upon degradation of the biodegradable material.

In some embodiments, the implant body includes a magnetic coating configured to attract the antibody-conjugated magnetic particles.

In some embodiments, the antibody-conjugated magnetic particles are configured to attach to the implant following circulation in the blood vessel in response to a magnetic field that redirects that antibody-conjugated magnetic particles to the implant.

In some embodiments, the antibody-conjugated magnetic particles are configured to selectively bind epCAM protein of the circulating tumor cells (CTC).

In some embodiments, the apparatus further includes an electromagnetic coil unit configured to generate one or more magnetic fields which are applied to the antibody-conjugated magnetic particles.

In some embodiments, an electromagnetic coil unit is configured to be implanted under skin of the subject.

In some embodiments, an electromagnetic coil unit is configured to externally coupled to skin of the subject.

In some embodiments, the apparatus further includes a computer processor configured to control generating of the magnetic field by the electromagnetic coil unit.

In some embodiments, the releasing element includes an injector configured to inject the antibody-conjugated magnetic particles into the subject's bloodstream.

In some embodiments, the apparatus further includes a computer processor configured to control injection of the antibody-conjugated magnetic particles into the subject's bloodstream at predetermined time intervals.

In some embodiments, in response to one or more magnetic fields applied to the antibody-conjugated magnetic particles, the antibody-conjugated particles are configured to detach from the implant body and circulate within a portion of the blood vessel that is under the magnetic field, while being confined to that portion of the blood vessel.

In some embodiments, the antibody-conjugated magnetic particles are configured to attach to the implant following circulation in the blood vessel in response to termination of the magnetic field.

There is further provided, in accordance with some embodiments of the present invention, apparatus for capturing circulating tumor cells (CTC) in vivo, the apparatus including:
an intravascular implant configured to be implanted in a blood vessel of a subject, the implant including an implant body configured to be positioned in the blood vessel; and
antibody-conjugated magnetic particles configured to (a) magnetically attach to the implant body, and (b) selectively bind circulating tumor cells (CTC) in the blood vessel,
wherein, in response to one or more magnetic fields applied to the antibody-conjugated magnetic particles, the antibody-conjugated particles are configured to detach from the implant body and circulate within a portion of the blood vessel that is under the magnetic field, while being confined to that portion of the blood vessel.

In some embodiments, the intravascular implant is configured to be implanted in a vena cava of the subject.

In some embodiments, the intravascular implant is configured to be implanted in a vein that carried blood from a tumor within the subject.

In some embodiments, the intravascular implant is configured to inactivate the CTC by applying shear stress to the CTC as blood flows through the intravascular implant.

In some embodiments, the antibody-conjugated magnetic particles are configured to attach to the implant body following circulation in the blood vessel in response to termination of the one or more magnetic fields.

In some embodiments, the antibody-conjugated magnetic particles are configured to attach to the implant body following circulation in the blood vessel in response to a magnetic field that redirects that antibody-conjugated magnetic particles to the implant.

In some embodiments, the implant body is shaped to define an outer portion and a central potion, and the implant body is configured such that when implanted in the blood vessel, the outer portion contacts inner walls of the blood vessel to anchor the implant body in the blood vessel, and the central portion is positioned in the lumen of the blood vessel.

In some embodiments, the antibody-conjugated magnetic particles are configured to selectively bind epCAM protein of the circulating tumor cells (CTC).

In some embodiments, the implant body includes a magnetic coating configured to attract the antibody-conjugated magnetic particles.

In some embodiments, the implant includes a biodegradable material that is degradable into the blood vessel, the antibody-conjugated magnetic particles are embedded in the biodegradable material, and the antibody-conjugated magnetic particles are configured to be released into the blood vessel upon degradation of the biodegradable material.

In some embodiments, the apparatus further includes an electromagnetic coil unit configured to generate the one or more magnetic fields which are applied to the antibody-conjugated magnetic particles.

In some embodiments, an electromagnetic coil unit is configured to be implanted under skin of the subject.

In some embodiments, an electromagnetic coil unit is configured to externally coupled to skin of the subject.

In some embodiments, the apparatus further includes a computer processor configured to control generating of the magnetic field by the electromagnetic coil unit.

In some embodiments, the apparatus further includes an injector configured to inject the antibody-conjugated magnetic particles into the blood vessel of the subject.

In some embodiments, the apparatus further includes a computer processor configured to control injection of the antibody-conjugated magnetic particles into the blood vessel of the subject at predetermined time intervals.

There is further provided, in accordance with some embodiments of the present invention, a method for capturing circulating tumor cells (CTC) in vivo, the method including:
implanting an intravascular implant in a blood vessel of a subject, such that an implant body of the intravascular implant is positioned in the blood vessel;
administering antibody-conjugated magnetic particles into the subject's blood, the antibody-conjugated magnetic particles being configured to (a) magnetically attach to the implant body, and (b) selectively bind circulating tumor cells (CTC) in the blood vessel; and
causing the antibody-conjugated particles to detach from the implant body and circulate within a portion of the blood vessel, while being confined to a portion of the blood vessel by applying one or more magnetic fields to the antibody-conjugated magnetic particles within the portion of the blood vessel.

In some embodiments, implanting the intravascular implant in the blood vessel of the subject includes implanting the intravascular implant in a vena cava of the subject.

In some embodiments, implanting the intravascular implant in the blood vessel of the subject includes implanting the intravascular implant in a vein that carried blood from a tumor within the subject.

In some embodiments, the intravascular implant is configured to inactivate the CTC by applying shear stress to the CTC as blood flows through the intravascular implant.

In some embodiments, applying one or more magnetic fields to the antibody-conjugated magnetic particles within the portion of the blood vessel includes causing the antibody-conjugated magnetic particles to attach to the implant body following circulation in the blood vessel in response to termination of the one or more magnetic fields.

In some embodiments, the method further includes causing the antibody-conjugated magnetic particles to attach to the implant body following circulation in the blood vessel by applying a magnetic field that redirects that antibody-conjugated magnetic particles to the implant.

In some embodiments, the implant body is shaped to define an outer portion and a central potion, and the implant body is configured such that when implanted in the blood vessel, the outer portion contacts inner walls of the blood vessel to anchor the implant body in the blood vessel, and the central portion is positioned in the lumen of the blood vessel.

In some embodiments, the antibody-conjugated magnetic particles are configured to selectively bind epCAM protein of the circulating tumor cells (CTC).

In some embodiments, the implant body includes a magnetic coating configured to attract the antibody-conjugated magnetic particles.

In some embodiments, the implant includes a biodegradable material that is degradable into the blood vessel, the antibody-conjugated magnetic particles are embedded in the biodegradable material, and the antibody-conjugated magnetic particles are configured to be released into the blood vessel upon degradation of the biodegradable material.

In some embodiments, the method further includes injecting the antibody-conjugated magnetic particles into the blood vessel of the subject.

In some embodiments, injecting the antibody-conjugated magnetic particles into the blood vessel of the subject includes controlling injection of the antibody-conjugated magnetic particles into the blood vessel of the subject such that the antibody-conjugated magnetic particles are injected into the blood vessel of the subject at predetermined time intervals.

There is further provided, in accordance with some embodiments of the present invention, a method for capturing circulating tumor cells (CTC) in vivo, the method including:
implanting an intravascular implantable implant body in a blood vessel of a subject; and
periodically releasing antibody-conjugated magnetic particles into a bloodstream of the subject, the antibody-conjugated magnetic particles being configured, upon being released into the subject's bloodstream, to (a) magnetically attach to the implant body, and (b) selectively bind circulating tumor cells (CTC) in the blood vessel, such as to replenish the implant body with antibody-conjugated magnetic particles.

In some embodiments, periodically releasing antibody-conjugated magnetic particles into the subject's bloodstream includes placing a biodegradable material into the blood vessel, the antibody-conjugated magnetic particles are embedded in the biodegradable material, and are configured to become released into the blood vessel upon degradation of the biodegradable material.

In some embodiments, the implant body includes a magnetic coating configured to attract the antibody-conjugated magnetic particles.

In some embodiments, the antibody-conjugated magnetic particles are configured to attach to the implant following circulation in the blood vessel in response to a magnetic field that redirects that antibody-conjugated magnetic particles to the implant.

In some embodiments, the antibody-conjugated magnetic particles are configured to selectively bind epCAM protein of the circulating tumor cells (CTC).

In some embodiments, in response to one or more magnetic fields applied to the antibody-conjugated magnetic particles, the antibody-conjugated particles are configured to detach from the implant body and circulate within a portion of the blood vessel that is under the magnetic field, while being confined to that portion of the blood vessel.

In some embodiments, the antibody-conjugated magnetic particles are configured to attach to the implant following circulation in the blood vessel in response to termination of the magnetic field.

In some embodiments, periodically releasing antibody-conjugated magnetic particles into the subject's bloodstream includes periodically releasing antibody-conjugated magnetic particles into the subject's bloodstream using an injector.

In some embodiments, periodically releasing antibody-conjugated magnetic particles into the subject's bloodstream using an injector includes using a computer processor to control injection of the antibody-conjugated magnetic particles into the blood vessel of the subject at predetermined time intervals.

There is further provided, in accordance with some embodiments of the present invention, an implantable medical device for eliminating circulating tumor cells (CTCs) in vivo including:
a first shaft region including:
a flexible magnetic core;
one or more side-emitting UV optical fibers; and
a plurality of magnetic particles bound to one or more biomarkers, wherein the plurality of magnetic particles are positioned over the optical fibers.

In some embodiments, the implantable medical device further includes an end protector.

In some embodiments, the end protector is located at the insertion end of the device, and is attached to the distal end of the first shaft region.

In some embodiments, the first shaft region further includes one or more, optionally a plurality of fins attached to the surface of the shaft region.

In some embodiments, the implantable medical device further includes one or more additional shaft region(s), and the first shaft region and any additional shaft regions are separated from each other by a spacer, such that a spacer is located between two adjacent shaft regions.

In some embodiments, the magnetic particles are substantially spherical and wherein the plurality of magnetic particles has a mean size in the range of 500 nm to 100 microns, from 0.1 mm to 1 mm, or from 1 mm to 10 mm.

In some embodiments, the magnetic particles include a material selected from the group consisting of ferromagnetic materials, such as neodymium, iron, nickel, cobalt, and gandolinium, and samarium, and magnetic alloys thereof.

In some embodiments, the one or more UV-optical fibers are capable of emitting UV radiation having a wavelength in the range of 280 nm to approximately 320 nm.

In some embodiments, the implantable medical device further includes a non-thrombogenic material or coating on the outer surface of the one or more side-emitting UV optical fibers, optionally, wherein the coating includes an anticoagulant, such as heparin.

In some embodiments, the biomarker is ligand that binds to a receptor on the CTCs.

In some embodiments, the implantable medical device further includes an attachment end located opposite the insertion end of the device.

In some embodiments, the attachment end is configured to attach to a plunger in an insertion device and/or is configured to attach to a connector for a plunger in an insertion device.

In some embodiments, the attachment end is configured to attach to a connector for a UV generator.

In some embodiments, the diameter of the first shaft region, and any additional shaft region(s), if present, is in the range of 0.010 to 0.040 inches (0.254 mm-1.016 mm), such as from 0.014 and 0.039 inches (0.36 mm-1.0 mm), optionally from about 0.030 inches to about 0.039 inches (0.76 mm-1.0 mm), such as from 0.035 inches to 0.039 inches (0.9 mm-1.0 mm). In some embodiments, the diameter of the first shaft region, and any additional shaft region(s), if present, is in the range of 10 to 13 mm.

In some embodiments, the diameter of the end protector is greater than the diameter of the first shaft region and smaller than the diameter of a small blood vessel, such as at least 10% greater than the diameter of the first shaft region and smaller than 3 mm.

There is further provided, in accordance with some embodiments of the present invention, a system for targeting and eliminating circulating tumor cells (CTCs) in vivo including the implantable device, and an insertion device.

In some embodiments, the insertion device is a syringe including a plunger, barrel, and needle.

In some embodiments, the implantable device and the needle are provided in a sterile package.

In some embodiments, at least the insertion end of the implantable device is inside the shaft of the needle.

There is further provided, in accordance with some embodiments of the present invention, a method for targeting and eliminating circulating tumor cells (CTCs), including inserting the implantable medical device in a subject's small blood vessel.

In some embodiments, the device is inserted into the subject by injecting the device through a syringe into a blood vessel of the subject, and following insertion, the insertion end of the device is located at a depth of about 1 to about 5 mm below the surface of the skin.

In some embodiments, the device is inserted into the subject by injecting the device through a syringe into a blood vessel of the subject, and wherein following insertion, the insertion end of the device is located at a depth of about 5 cm to about 15 cm below the surface of the skin.

In some embodiments, the blood vessel is located in the subject's wrist or lower arm. In some embodiments, the blood vessel is located in the subject's leg, such as the femoral vein.

In some embodiments, during the insertion step, the device is inserted in the blood vessel in a direction opposite the blood flow, such that the blood pushes against the insertion end of the device.

In some embodiments, the method further includes:
attaching the attachment end of the device to a UV generator; and
periodically illuminating the accumulated circulating tumor cells with UV for a sufficient period of time to destroy circulating tumor cells that are attached to the device.

In some embodiments, two or more of the implantable medical devices are inserted into different blood vessels in the subject.

In some embodiments, the method further includes following 1-7 days, 1 week, 2 weeks, or 3 weeks following insertion, the implantable medical devices are removed.

In some embodiments, the method further includes, after removal, inserting another implantable medical device at the same or different insertion site.

The present invention will be more fully understood from the following detailed description of applications thereof, taken together with the drawings, in which:

### BRIEF DESCRIPTION OF THE DRAWINGS

FIGS. 1A, 1B, and 1C are schematic illustrations of a circulating tumor cells (CTC)-capturing implant configurations for selective binding of CTC from a blood stream of a subject, in accordance with some applications of the present invention;

FIGS. 2A and 2B are schematic illustrations of a CTC-capturing implant implanted in a blood vessel of a subject, in accordance with some applications of the present invention;

FIG. 3 is a schematic illustration of an additional configuration of a CTC-capturing implant, in accordance with some applications of the present invention;

FIG. 4 is a schematic illustration showing replenishing of the CTC-capturing implant by injection into a blood vessel of antibodies for selective binding of CTC, in accordance with some applications of the present invention;

FIG. 5 is a schematic illustration of a CTC-capturing implant, a portion of which is configured to undergo biodegradation to release viable antibodies for selective binding of CTC, in accordance with some applications of the present invention;

FIGS. 6A and 6B are schematic illustrations of side cross-sectional and front cross-sectional views respectively, of an implantable medical device, in accordance with some applications of the present invention;

FIGS. 7A and 7B are schematic illustrations of side cross-sectional and front cross-sectional views, respectively, of an implantable medical device following implantation, showing circulating tumor cells (CTC) in the blood stream in which the device is located, where one of the CTC is passively captured and attached to a biomarker on an outer surface of the implantable medical device, in accordance with some applications of the present invention;

FIG. 7C is a schematic illustration depicting a slice of a portion of an implantable device, showing a tubular magnetic core in the center, surrounded by one or more side illuminating optical fibers, coated with an anticoagulant, in accordance with some applications of the present invention;

FIGS. 8A and 8B are schematic illustrations of side cross-sectional and front cross-sectional views, respectively of an implantable medical device following implantation in a subject's small blood vessel, in accordance with some applications of the present invention;

FIG. 9 is a cross sectional view of a schematic containing the implantable device depicted in FIGS. 6A and 6B inside a syringe needle and enclosed in a sterile package, in accordance with some applications of the present invention;

FIG. 10 is a cross-sectional view of a schematic containing the implantable device depicted in FIGS. 6A and 6B inside a syringe needle and attached at its distal end to a syringe piston, in accordance with some applications of the present invention;

FIG. 11 is a cross-sectional view of a schematic of the implantable device depicted in FIGS. 6A and 6B following subcutaneous insertion into a small blood vessel in a subject with the needle inside the subject, in accordance with some applications of the present invention;

FIG. 12 is a schematic illustration of a cross-sectional view of the implantable device depicted in FIGS. 6A and 6B following subcutaneous insertion, which is in electrical communication with an external UV generator, in accordance with some applications of the present invention;

FIG. 13A depicts an embodiment of the implantable device shown with a plurality of hemispherical fins attached to the outer surface of the device, in accordance with some applications of the present invention;

FIG. 13B is a schematic illustration of the same device as depicted in FIG. 13A showing the movement of the device from one side to the opposite side (depicted with dashed lines) within the blood vessel when subjected to the blood flow, in accordance with some applications of the present invention;

FIGS. 14A, 14B, and 14C depict an implantable device with a lima-bean shaped end protector at the insertion end, in accordance with some applications of the present invention; and

FIGS. 15A and 15B depict an implantable device implanted in a blood vessel, such as the femoral vein, in a manner that the blood flow is directed upstream of the insertion end of the device, in accordance with some applications of the present invention.

### DETAILED DESCRIPTION OF APPLICATIONS

In accordance with some applications of the present invention, systems, apparatus and methods are provided for capturing and destroying circulating tumor cells (CTC) in a subject in vivo. The systems, apparatus and methods provided herein involve implantable devices for capturing CTC in a subject in vivo. For some applications, systems and/or kits for delivering to the subject of the implantable devices for capturing CTC in the subject in vivo, are provided. For some applications, systems for use with implantable devices for capturing and destroying CTC in a subject in vivo, are provided.

### Exemplary Implantable Devices and Methods of Using

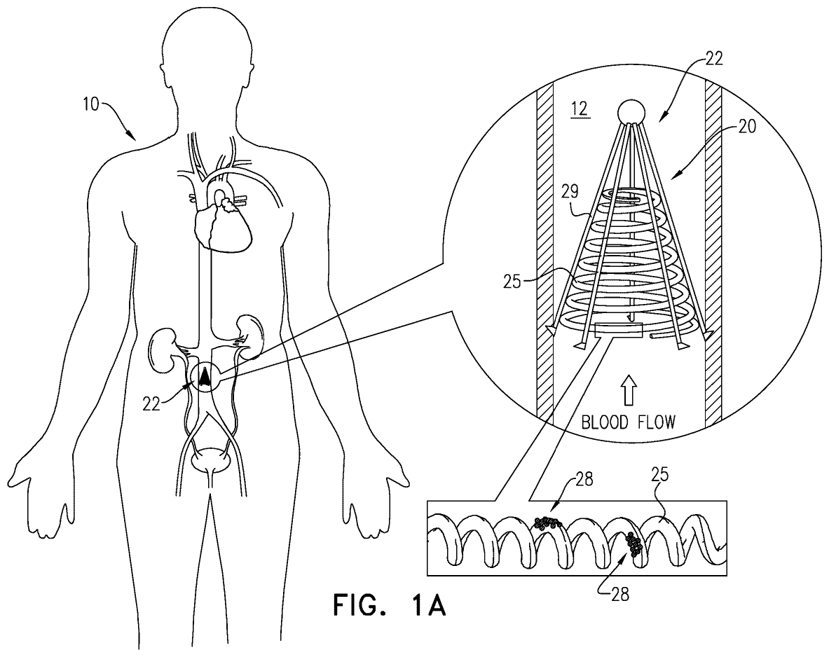

Reference is made to FIGS. 1A-5, which are exemplary implantable devices and methods of using, in accordance with some applications of the present invention.

Reference is first made to FIGS. 1A, 1B, and 1C, which are schematic illustrations of optional configurations of an apparatus comprising a circulating tumor cells (CTC)-capturing implant 22, in accordance with some applications of the present invention. Implant 22 is typically introduced into a blood vessel of a subject and remains deployed in the blood vessel for trapping and inactivating blood-borne circulating tumor cells (CTC) in vivo. By removing the CTC from the blood circulation and rendering the CTC harmless, the risk of metastasis incubation and formation in subjects with cancer is generally reduced.

For some applications, implant 22 typically comprises an implant body 20 and a biological CTC-capturing component. The implant body typically comprises a metallic and/or a polymeric biocompatible implant body structure that is anchored in the blood vessel and provides structural support for the biological CTC-capturing component. The CTC-capturing components may comprise any suitable biomarker from the description provided hereinbelow. Typically, the CTC-capturing components of implant 22 comprise antibodies for selectively targeting antigens expressed by the CTC.

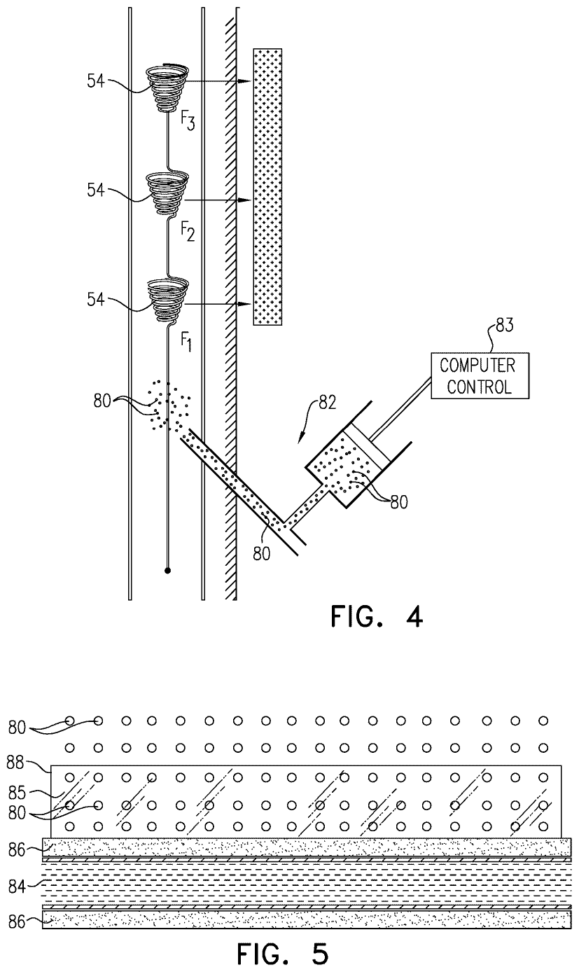

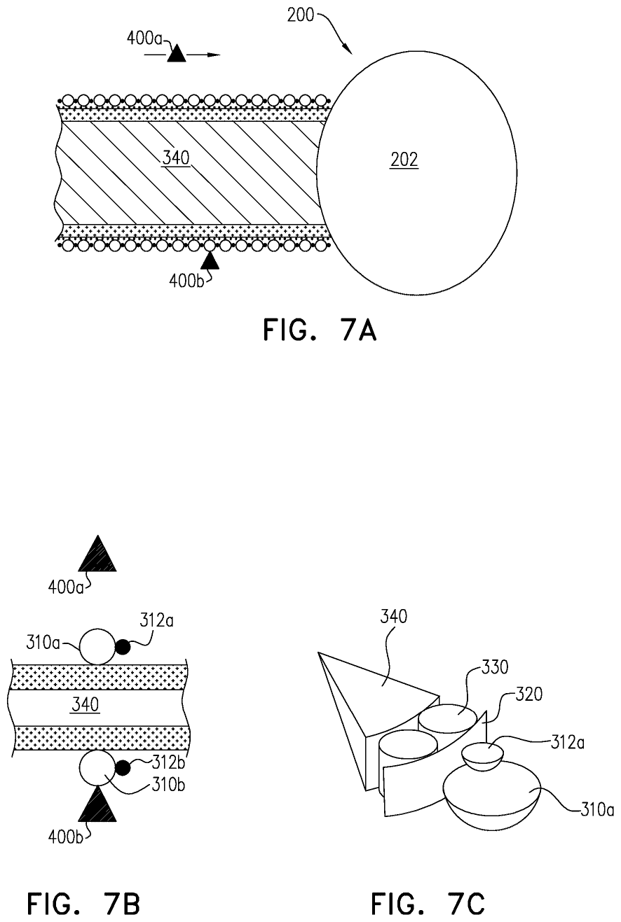

In accordance with some applications of the present invention, selective detection and enrichment of the CTC from the bloodstream is generally based on utilizing unique surficial antigen expression of the CTC to distinguish the CTC and facilitate their isolation from within large numbers of leukocytes, erythrocytes, and other blood components. A common surface molecule on CTC is the epithelial cell adhesion molecule (EpCAM), which originates from the epithelium. EpCAM is a transmembrane glycoprotein that is present in 80% of solid cancers (such as breast, colorectal, and prostate cancer), but is absent from peripheral blood cells. For some applications, the CTC-capturing component in implant 22 is a CTC-capturing antibody 28, e.g., anti-EpCAM configured to selectively bind to EpCAM in the CTC. For some applications, CTC-capturing antibody 28 is part of a magnetic particle-antibody complex, e.g., as shown in in FIG. 7B, which shows a biomarker 312a/b (e.g., an antibody) that is conjugated to a magnetic particle 310a/b, to form an antibody-conjugated magnetic particle. A magnetic particle-antibody complex is also indicated, for example, by a schematic representation in FIG. 4, labelled by reference numeral 80.

Additionally, or alternatively, in accordance with some applications of the present invention, the CTC are captured and isolated based on technologies that utilize the physical properties of the CTC, e.g., size, density, and capacitive character.

FIGS. 1A, 1B, and 1C, show various non-limiting possible configurations of CTC-capturing implant 22 for deployment in a blood vessel of the subject, comprising implant body 20 and CTC-capturing antibodies 28, in accordance with some applications of the present invention.

FIG. 1A is a schematic illustration showing CTC-capturing implant 22 implanted in a blood vessel 12 of a subject 10, in accordance with some applications of the present invention. As shown, for some applications, implant 22 is implanted in an inferior vena cava of the subject. FIG. 1A shows an optional configuration of implant 22 comprising implant body 20 (e.g., a wire scaffold) and CTC-capturing antibodies 28, which are infused into the implant body as will be described in further detail hereinbelow. As shown by way of illustration and not limitation, implant body 20 is structured such that outer portions 29 at least partially contact inner walls of blood vessel 12 to anchor implant 22 in the blood vessel, and a central helical portion 25 is positioned in the lumen of the blood vessel.

In other applications, implant 22 is shaped to define one or more coils 24 (e.g., nitinol coils) as shown in FIG. 1B. Implant 22 may comprise any suitable number of coils 24, e.g., 3-4 coils, that together form the structural component of implant 22, which is deployed within the blood vessel. For some applications, coil 24 is anchored to walls of the blood vessel via one or more anchoring elements 26.

In yet other applications, implant 22 is shaped to define a three-dimensional structure as shown in FIG. 1C. For some such applications, the three-dimensional structure comprises multiple wires (e.g., nitinol wires), or multiple struts. For some such applications, the implant is positioned in the blood vessel such that a portion of the wires are positioned against the inner walls of the blood vessel to anchor the implant in the blood vessel.

Typically, the various optional configurations of implant 22 that are shown in FIGS. 1A, 1B and 1C are designed to increase a surface area of the implant to maximize the blood flow that contacts the implant, thereby increasing the CTC-capturing potential of implant 22. For some applications, implant 22 is deployed in the blood vessel at angular positions that increase the surface area of the implant, and thus increasing the flow of blood that contacts the implant. Additionally, implant 22 is typically structured and positioned such that it generally does not interfere (and does not block) blood flow, but rather allows the blood to flow therethrough and around the implant so as to maximize the areas of the implant that come in contact with the blood flowing in the blood vessel.

For some applications, implant 22 is shaped to define any other configuration that increases the surface area of the implant, such that the blood-facing surface area of the implant is increased. For example, for some applications, implant 22 is shaped to define a honeycomb shaped implant (not shown). In such a manner, the surface area of the implant is relatively large, allowing for a relatively large area of the implant to be in contact with the blood flow in the blood vessel, thereby increasing the CTC-capturing potential of implant 22. For some such applications, in which implant 22 is shaped to define a honeycomb structure, implant 22 is made of a thin and easily foldable material (e.g., 0.1 mm in thickness), such that the implant can be inserted into a relatively small insertion device, e.g., a 0.5-3 mm insertion device e.g., a 1-3, e.g., a 2 mm insertion device for implantation in the blood vessel.

In general, the various configurations of implant 22 described herein are configured such that the implant can be delivered into the blood vessel by injection. Typically, implant 22 is easily collapsible, e.g., folded, into an injection device for delivery into the blood vessel. Once in the blood vessel, implant 22 assumes an expanded state that anchors the implant against the inner walls of the blood vessel for securing the implant in the vessel for a desired period of time (e.g., days, weeks or months).

For some applications, the internal structure of the implant (e.g., an internal structure as shown in FIG. 1B, or FIG. 1C, an internal honeycomb structure and/or any other internal structure described herein) is configured to apply shear stress to the CTC that are within the bloodstream as blood flows through the internal structure of the implant.

Optionally but not necessarily, implant 22 is positioned within a major blood vessel of the subject in order to maximize exposure of implant 22 to the blood circulation of the subject. For example, implant 22 is configured to be positioned within a major artery of the subject, such as an aorta. For some such applications, implant 22 is structured such as to withstand the blood pressure in the artery. Alternatively, or additionally, implant 22 is implanted in a major vein of the subject, e.g., a vena cava of the subject (e.g., an inferior vena cava as shown in FIG. 1A). Since the velocity peaks and pressure are lower in the vena cava compared to the aorta, the probability of capturing CTC by implant 22 is greater in the vena cava than in the aorta. Thus, in accordance with some applications of the present invention, implant 22 is implanted in the vena cava of the subject, thereby increasing the CTC-capturing optional of implant 22 leading to a reduced risk of the cancer metastasizing through circulating CTC.

Typically, implant 22 is implanted in a major blood vessel for an extended time period (e.g., days, weeks and/or months), thereby increasing exposure of implant 22 to the subject's blood flow. Additionally, or alternatively, implant 22 is implanted in small blood vessels of the bloodstream of the subject. Further additionally or alternatively, implant 22 is implanted in the lymphatic system of the subject.

As described hereinabove, CTC-capturing implant 22 as shown in FIGS. 1A-1C, typically comprises CTC-capturing antibodies 28 that selectively bind to CTC in the bloodstream and remove them from the blood circulation of the subject. In such a manner, CTC circulating with blood that passes through implant 22 are attached to the antibodies in implant 22 typically through proteins expressed by the CTC (e.g., epCAM) and that are targeted by the antibodies. It is noted that antibodies targeting epCAM are provided by way of illustration and not limitation. It is noted the antibodies for targeting CTC provided in accordance with some applications of the present invention, may target any other cancer-specific protein (in any type of cancer) expressed by the CTC (or any protein expression of which is increased by the CTC compared to healthy cells). (It is noted that FIG. 1A shows a schematic representation of CTC-capturing antibodies 28. It is to be understood that implant 22 as shown in all FIGS. 1B-1C also comprises CTC-capturing antibodies 28, although they are not schematically illustrated in FIGS. 1B-C.) For some applications, the CTC-capturing antibodies are impregnated on implant 22. Typically, the antibodies are immobilized onto implant 22 (specifically onto implant body 20), that is anchored in a stationary manner in the blood vessel. For some such applications, binding of the CTC to the antibodies is caused by impact of the CTC with the antibodies in implant 22. Impact attachment of the CTC to the antibodies is caused when CTC moving through the bloodstream pass by/through the stationary implant positioned in the vasculature of the subject and collide with the antibodies in the implant. When captured by the antibodies, the CTC are typically trapped in the implant, inactivated and rendered harmless. Typically, when captured, the CTC are destroyed by processes such as immune attacks, shear stress, anoikis, oxidative stress and the lack of cytokines and growth factors.

### Attachment of CTC to Free-Floating Antibodies in the Bloodstream

In some cases, it may be advantageous for the CTC-capturing antibodies to float freely in the bloodstream, rather than to be immobilized on a stationary implant in the blood vessel. In such a manner, the likelihood of the CTC-capturing antibodies pairing with the CTC is generally increased. However, it is generally not desirable for the CTC-capturing antibodies to circulate extensively and uncontrollably in the blood circulation for an extended period of time. Therefore, in accordance with some applications of the present invention, apparatus and methods are provided for allowing floating of the CTC-capturing antibodies within a defined “closed” segment of the blood vessel. The defined “closed” segment of the blood vessel refers to a portion in the blood vessel that is under a magnetic force that allows motion of the CTC-capturing antibodies within the defined, predetermined segment of the vein, but prevents the CTC-capturing antibodies from leaving the defined segment to circulate freely and extensively within the blood circulation. In such a manner, the CTC-capturing antibodies travel within a selected predefine area of the bloodstream but are prevented by a magnetic field from continuing their systemic journey.

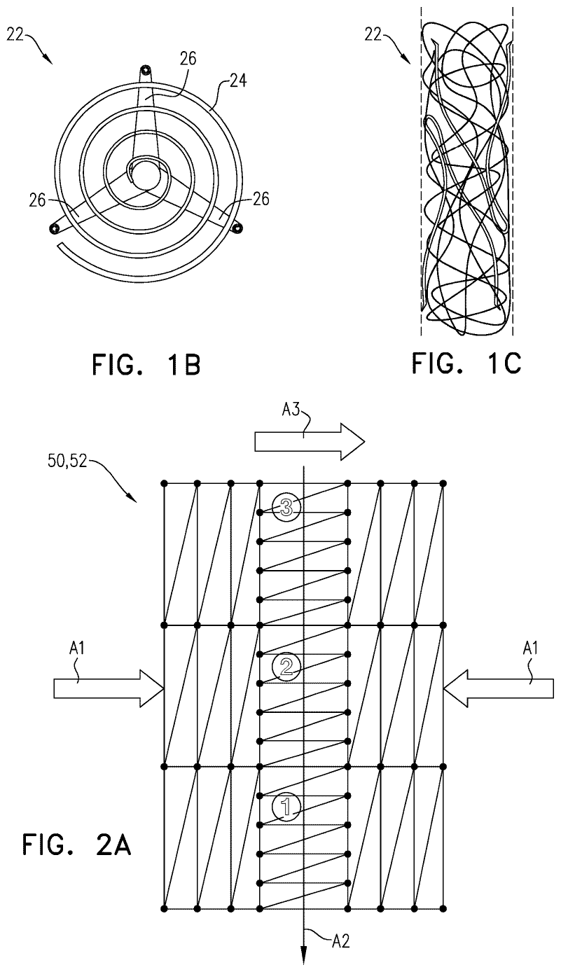

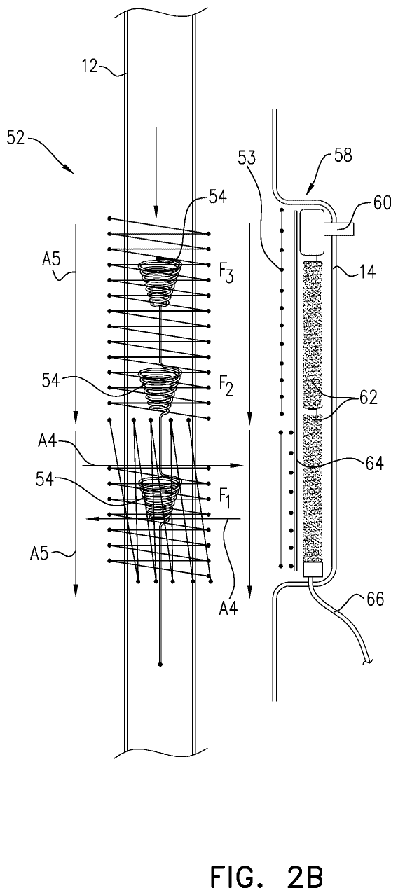

Reference is now made to FIGS. 2A and 2B, which are schematic illustrations of configurations and operation of an apparatus 50 comprising a circulating tumor cells (CTC)-capturing implant 52, in accordance with some applications of the present invention. Implant 52 typically comprises a magnetic implant that is similar to implant 22, implant 52 being positioned in a blood vessel of a subject. Apparatus 50 additionally comprises magnetic CTC-capturing antibodies. The magnetic CTC-capturing antibodies are typically formed from magnetic particles that are conjugated to CTC-capturing antibodies (e.g., magnetic nanoparticles (MNP) that are an antibody-modified nanomaterial, by having an antibody conjugated thereto. Typically, the antibody is an antibody for selectively binding epCAM, as described herein). The magnetic particle-antibody complex is indicated, for example, by a schematic representation in FIG. 4 and labelled by reference numeral 80. Another schematic representation of a magnetic particle-antibody complex is indicated, in FIG. 7B, which refers to a biomarker 312a/b (e.g., an antibody) that is conjugated to a magnetic particle 310a/b, to form an antibody-conjugated magnetic particle.

Typically, the magnetic CTC-capturing antibodies remain attached to the magnetic implant within the blood vessel of the subject. Application of a magnetic field causes detachment of the magnetic CTC-capturing antibodies from the implant and motion of the antibodies in the blood vessel, within a defined area of the blood vessel. Other magnetic fields cause return of the magnetic CTC-capturing antibodies to the implant.

In FIG. 2A, areas in which segments of circulating tumor cells (CTC)-capturing implant 52 are positioned in the blood vessel, are indicated by numbers 1, 2, 3. Upon application of a lateral magnetic field (indicated by arrows A1), magnetic CTC-capturing antibodies that are attached to the implant are detached from the implant (which is also magnetic) and are allowed to travel in the blood vessel until they are blocked by a second lateral magnetic field (indicated by arrow A3). By applying an axial magnetic field opposite to the bloodstream, the antibodies are redirected back to the implant. (The axial magnetic field is indicated by arrow A2). In such a manner, if CTC successfully bind to the antibodies, they remain within the closed segment of the blood vessel and are eventually destroyed. Typically, when captured, the CTC are destroyed by processes such as immune attacks, shear stress, anoikis, oxidative stress and the lack of cytokines and growth factors.

FIG. 2B is a schematic illustration of apparatus 50 implanted in blood vessel 12 (e.g., a vena cava of the subject), in accordance with some application of the present invention. As shown, for some applications, apparatus 50 comprises implantable magnetic coils 54, each coil 54 implanted in an area of the blood vessel indicated as F1, F2 and F3. (The coils are examples of implant bodies, as described hereinabove.) As described hereinabove, apparatus 50 additionally comprises magnetic CTC-capturing antibodies that are influenced by magnetic fields applied thereto. Magnetic CTC-capturing antibodies are typically attached to implantable coils 54, unless a magnetic force that is applied to the antibodies causes detachment of the antibodies from the coils and circulation of the antibodies in the blood vessel (typically within and between areas F1-F3). As shown in FIG. 2B, apparatus 50 additionally comprises an electromagnetic coil unit 58 configured to be implanted directly under skin 14 of the subject or on the skin of the subject (and for example, strapped on with a bandage), and configured to generate magnetic fields which are applied to the magnetic CTC-capturing antibodies.

Electromagnetic coil unit 58 typically comprises one or more electromagnetic coils 53, a rechargeable battery 62, a control computer circuit 64, and, optionally, a cable 66 extending externally to the subject. Additionally, an on/off switch 60 which is positioned transcutaneously is controllable by a user to initiate or terminate generation of the magnetic field. Application of alternating magnetic fields (indicated by arrows A4) by electromagnetic coil unit 58 causes and controls motion of the CTC-magnetic capturing antibodies such that they are allowed to circulate within areas F1-F3, however magnetic force generally does not allow for the antibodies to continue to travel in the bloodstream outside of areas F1-F3.

Application of a pulling magnetic field (indicated by arrows A5) redirects the CTC-magnetic capturing antibodies back to coils 54. In such a manner, if CTC successfully bind to the antibodies, they remain within the closed segment (area F1-F3) of the blood vessel and are eventually destroyed. Typically, when captured, the CTC are destroyed by processes such as immune attacks, shear stress, anoikis, oxidative stress and the lack of cytokines and growth factors.

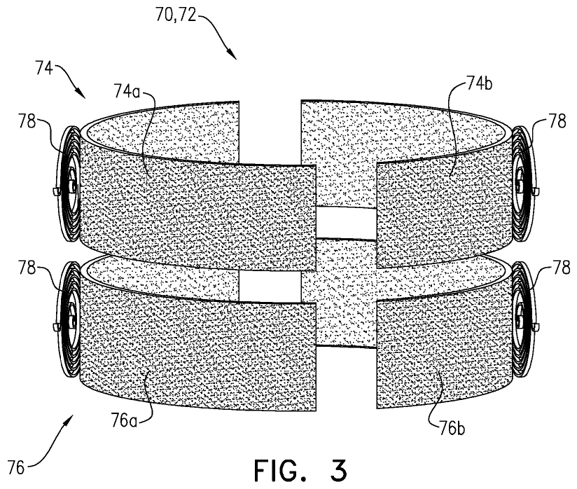

Reference is now made to FIG. 3, which is a schematic illustration of an apparatus 70 for capturing circulating tumor cells (CTC), in accordance with some applications of the present invention. In accordance with some applications of the present invention, apparatus 70 is configured to provide a defined “closed” segment of the vein, as described hereinabove, in which the CTC-magnetic capturing antibodies are detached (by a magnetic force) from the implant and allowed to circulate within the defined segment in the vein, but are prevented (by a magnetic force) from escaping the defined segment and circulating within the entire body.

For some applications, apparatus 70 comprises an implant 72 configured to be deployed in a blood vessel of a subject, e.g., the vena cava. Apparatus 70 additionally comprises magnetic CTC-capturing antibodies that attach and detach from implant 72 depending on the magnetic forces applied thereto.

Implant 72 comprises implant sections 74 and 76 that are spaced apart and deployed in separate locations across the blood vessel (typically, implant sections 74 and 76 are separated in the blood vessel by a distance of 0.1-20 cm, e.g., 1-10 cm). Generally, magnetic attraction and repulsion of the magnetic CTC-capturing antibodies causes motion of the magnetic CTC-capturing antibodies between implant sections 74 and 76 thereby allowing the CTC-capturing antibodies to circulate within a defined area of the blood sample. In such a manner, the likelihood of capturing CTC by the antibodies increases while at the same time controlling the area of the bloodstream in which the antibodies circulate.

For some applications, each one of implant sections 74 and 76 comprises two half ring portions 74a/b and 76a/b, respectively. Each one of portions 74a/b and 76a/b comprises a ferromagnetic material that attracts the magnetic CTC-capturing antibodies to sections 74 and 76. Additionally, each portion 74a/b and 76a/b comprises an antenna 78 configured to receive an electrical field and, in response, to generate a magnetic field that causes the implant section 74 to release the antibodies that are attached thereto. Simultaneously, section 76 is activated to attract the antibodies such that CTC-capturing antibodies are attracted across the blood vessel. As a result, by moving the CTC-capturing antibodies from section 74 to section 76, the blood vessel cross-section is covered with free-floating antibodies, thereby increasing the potential interaction between CTC in the blood stream and the CTC-capturing antibodies.

For some applications, half rings portions 74a/b and 76a/b are activated sequentially by simultaneously applying a magnetic force of repulsion to half ring portion 74a and a magnetic attraction force to half ring section 76b. For some applications, the same procedure is repeated with respect to half ring section 74b and half ring section 76a. Typically, each antenna 78 has a unique frequency for receiving the externally generated electrical field, this allowing selective activation.

### Replenishing

Reference is made to FIG. 4 and FIG. 5, which are schematic illustrations of apparatus and methods for replenishing the CTC-capturing antibodies. Since antibodies in the body expire overtime (typically having a half-life of several days), it is required to replenish the antibodies in order to maintain the CTC-capturing implants provided herein in a viable and operative condition.

FIG. 4 depicts replenishment of the CTC-capturing antibodies by direct injection into the blood vessel. For example, FIG. 4 shows replenishment of the magnetic CTC-capturing antibodies 80 by intravenous injection into the vena cava using an injector 82 while simultaneously applying a magnetic field to prevent the antibodies from spreading throughout the body. (Antibodies 80 are typically not allowed to freely-circulate beyond implant coil 54 in area F3). For some applications, a computer processor control unit 83 controls automatic periodic delivery of antibodies 80 at predetermined time intervals, e.g., every 4-6 days. For some applications, the implant coil is made of a magnetic material such that the implant attracts the antibodies without a magnetic field being applied.

FIG. 5 is schematic illustration of an example of a CTC-capturing implant (CTC-capturing implant 52 described herein) comprising a CTC-capturing antibodies replenishing layer 88. For the applications described in FIG. 5, replenishing of the CTC-capturing antibodies is provided in vivo by the implant itself, rather than being administered through an external injection device, as described with reference to FIG. 4.

FIG. 5, shows a cross section of a CTC-capturing implant structure (such as implant 52) comprising nitinol wires 84 that are coated by a magnetic coating 86. (The nitinol wires are examples of implant bodies, as described hereinabove.) Additionally, the implant structure includes magnetic CTC-capturing antibodies 80 that are attracted to the magnetic coating on the nitinol wires. CTC-capturing antibodies 80 are distributed within a biodegradable material 85 in layer 88. The biodegradable material gradually degrades in the body thereby periodically exposing viable antibodies that are able to emerge from the implant to pair with the CTC.

### Diagnosis

Reference is again made to FIGS. 1A-15B.

Typically, detection of CTC in the blood, and quantification of the CTC is evaluated for diagnostic purposes, e.g., for determining a prognosis of a subject and deciding on an appropriate treatment plan.

Thus, in accordance with some applications of the present invention, apparatus and methods provided herein can be used for diagnostic purposes by, e.g., by detecting a number of CTC that is captured by the implants described herein. Thus, the implantable devices described herein are configured for both diagnostic purposes (by assaying the condition of the subject by counting the number of captured CTC) and treatment purposes (by removing the CTC from the blood circulation). Additionally, an efficacy of treatment can be assessed.

### Implantable Devices and Methods for Denaturing Circulating Tumor Cells (CTC)

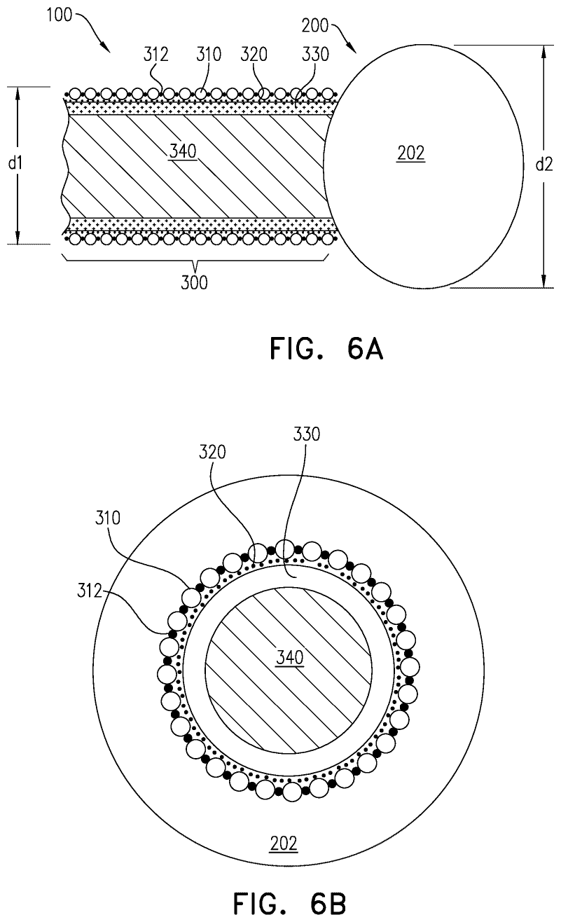

Reference is now made to FIGS. 6A-15B, which are schematic illustrations of implantable devices 100 for capturing and denaturing the CTC in vivo, and devices for use with the implantable devices. The following description is a general overview of the implantable devices for capturing CTC in situ to immobilize the CTC on the surface of the device and prevent them from circulating and metastasize at another site in the subject's body, using denaturing UV light as disclosed herein with reference to FIGS. 6A-15B. In some applications, the apparatus and methods described with reference to FIGS. 6A-15B are combined with apparatus and methods described with reference to FIGS. 1A-5. It is noted that the terms “implant” (which is typically used with reference to FIGS. 1A-5) and “implantable device” or “device” (which are typically used with reference to FIGS. 6A-15B) are used interchangeably in the present application.

### Implantable Devices

For some applications, implantable devices 100 described herein are readily delivered via injection, such as via subcutaneous injection or intravenous injection, and remain in the subject for a prolonged period of time are described herein. While in the subject, CTC that flow past the devices attach to the one or more biomarkers that are on the surface of the device. In some applications, after a period of time, optionally at regular time intervals, such as daily, or as needed, the device is placed in electrical communication with a UV generator and deliver a sufficient amount of UV energy in the region in which the device is implanted in the subject's body via side illuminating fiber optics for a sufficient period of time to denature the attached CTC. The time period for side illumination with UV energy is typically short, such as for up to 30 seconds, up to 1 minute, up to 2 minutes, or up to 5 minutes.

### Structure

For some applications, the implantable devices 100 contain one or more shaft regions 300a, 300b that include a central tubular magnetic, flexible core 340, such as in the form of a wire or filament. A plurality of magnetic particles 310 are attached to one or more biomarkers 312 and are attached to the outer surface of the wire. The particles are connected to the central magnetic core via magnetic forces. The device terminates at its insertion end with an end protector 202. At its opposite end, the device terminates with an attachment end 220.

In some applications, one or more of the shaft regions is a UV emission region, in which the central magnetic flexible core is surrounded by optical fibers 330 that are able to transmit ultraviolet (UV) light (e.g. wavelengths in the range of about 10 nm to about 400 nm). The outer surface of the optical fibers is formed of a non-thrombogenic material or is coated with a non-thrombogenic coating and/or an anti-coagulant, such as heparin. As described above, the biomarker containing particles are attached to the outer surface of the wire via magnetic forces. The overall length of the device is selected based on the function of the blood vessel In which the device will be implanted. For devices that are configured to be implanted in blood vessels having diameter, at rest, in the range of about 10 to 13 mm, such as the femoral vein, the length of the device that is typically implanted in the blood vessel typically ranges from about 5 cm to about 11 cm. For devices that are suitable for implantation in blood vessels having diameters in the range of about 3 to 5 mm, such as small blood vessels in a subject's wrist or lower arm, the length of the device that is typically implanted in the blood vessel typically ranges from about 1 mm to about 5 mm.

The total diameter (dl) of the shaft region is generally in the range of about 0.010 to 0.040 inches (0.254 mm- 1.016 mm), such as from 0.014 and 0.039 inches (0.36 mm- 1.0 mm), optionally from about 0.030 inches to about 0.039 inches (0.76 mm- 1.0 mm), such as from 0.035 inches to 0.039 inches (0.9 mm- 1.0 mm).

In some applications, the shaft region(s) of the device has a wider diameter, such as 10 to 13 mm. Devices with shaft regions having diameters in this range are generally longer, such as 5 cm to about 11 cm, and are suitable for insertion in larger blood vessels, such as the femoral vein.

Optionally, one or more fins 212, such as a plurality of fins, are attached at different locations to the outer surface of the shaft region. The fins do not emit energy.

The device also contains one or more non-energy emission regions, such as end protector 202 at the insertion end 200, attachment end 220, and optionally one or more spacers 210.

The diameter (d2) of each of the one or more non-energy-emission regions is generally at least 10% greater, at least 20% greater, at least 30% greater, at least 40% greater, at least 50% greater, at least 60% greater, such as between about 50% and 70% greater, or between about 60% and 70% greater, or larger, than the diameter (d1) of the UV emission regions and is smaller than the inner diameter (d3) of a small blood vessel having a diameter in the range of 3-5 mm. Optionally the diameter (d2) is 1.5-3 times greater, such as 1.5-2 times greater, than the diameter of the diameter (d1) of the UV emission region. When more than one non-energy emission region is present, the diameters for each of the non-energy emission regions can be the same or different. The end protector in the insertion end can have any shape as long as the tip is rounded. If the end protector is in the shape of a sphere typical diameters are in the range of about 1.2 mm to about 2.0 mm, such as 1.3-1.8 mm, such as 1.5 mm. If the end protector has a shape with two or more different diameters, the largest diameter is typically in the range of about 1.2 mm to about 2.0 mm, such as 1.3-1.8 mm, such as 1.5 mm. Suitable diameters for the one or more spacers, are in the range of 1.2 to 2.0 mm, such as 1.3-1.8 mm, such as 1.5 mm.

The insertion end 200 of the device typically contains non-magnetic end protector 202, which has a greater diameter than the diameter of the diameter of the core with optical fiber surrounding it. Optionally, one or more spacers 210, each of which has a diameter greater than the diameter of the diameter of the core with optical fiber surrounding it are also located in one or more positions along the length of the core.

In some applications, the device contains one or more fins 212, such as two or more fins, or a plurality of fins, attached to or protruding from the outer surface of the device, such as the outer surface of the core.

The end opposite insertion end 200 of the device contains an attachment region configured to attach to a syringe piston or another insertion device to push the device into the desired position in the subject's body. Following insertion, the piston is separated from the attachment mechanism, and the attachment region is outside the subject's body. The attachment region is also configured to attach to and be removable from an external UV generator.

### Central Magnetic Core

Flexible core 340 of the implantable device includes a magnetic wire or filament, typically in the center of each shaft region 300.

Suitable materials for the magnetic filament include but are not limited to an axial flexible neodymium magnet.

The magnetic filament provides a magnetic field along its length axis that is sufficient to attract to the surface of the wire the nanoparticles without the use of an external magnetic source. The flexible magnetic filament can generate magnetic field gradients in the range of 100 to 10,000 T/m.

The magnetic filament has a suitable diameter and flexibility to allow it to be introduced into and retrieved from a blood vessel without causing occlusion or trauma. For example, the diameter is similar to the diameter of medical guidewires used in vascular interventional procedures. Suitable diameters for the flexible magnetic filament include about 0.010 to 0.040 inches (0.254 mm-1.016 mm), such as from 0.014 and 0.039 inches (0.36 mm-1.0 mm), optionally from about 0.030 inches to about 0.039 inches (0.76 mm-1.0 mm), such as from 0.035 inches to 0.039 inches (0.9 mm-1.0 mm).

### Side-Emitting UV Fiberoptics

In applications in which the shaft region is, or one or more of the shaft regions are, also a UV emission region, the outer surface of the central magnetic core is attached to one or more side-emitting UV optical fibers 330. Optionally more than one side-emitting UV optical fiber is attached to the outer surface of the central magnetic core, such as up to about 50, from 2-50, 10-50, 20-50, 2-40, 10-40, 20-40, 2-30, 10-30, 20-30, 2-20, 10-20, or from 2-10 side-emitting UV optical fibers.

Side-emitting UV optical fibers are able to emit UV light along their length to denature any CTC that are attached to the device.

Thin and flexible nano-enabled side-emitting UV optical fibers are light delivery devices that enable disinfection or photocatalytic oxidation by radiating UV light from light-emitting diodes (LEDs). Typically, the UV light irradiated by the side-emitting UV optical fiber has wavelengths in the range of 10 nm to about 400 nm, optionally the wavelengths are in the range of about 280 nm to about 320 nm. For many types of CTC, UV having a specific wavelength, such as in the range of approximately 280 nm to approximately 320 nm, is used to eradicate the CTC. Optionally, the side-emitting UV optical fibers administer a specific wavelength, which is selected based on the particular CTC in the subject's blood flow.

Optionally, the optical fibers are coated with a hemocompatible material or coating. Suitable hemocompatible, non-thrombogenic materials include but are not limited to polyethylene oxide (PEO), phosphorylcholine (PC), poly 2-methoxyethylacrylate, and triblock surface-modifying additives.

Side-emitting optical fibers may be formed by removing the peripheral cladding of conventional fiber-optic cord. For example, side emitting optical fibers can be formed from optical fibers in which defects have been introduced periodically in the fiber cladding to allow UV wavelengths from the core to partially escape along the fiber length. Other techniques to enable side-emission of light are also acceptable.

The one or more side-emitting UV optical fibers have a suitable diameter to permit insertion of the device in a subject's superficial blood vessels. Suitable diameters for each side-emitting UV optical fiber are typically less than 0.1 mm, such as in the range of 0.005 mm to 0.03 mm, in the range of about 0.01 mm to about 0.02 mm, optionally the diameter of the side-emitting UV optical fibers is about 0.01 mm. Side-emitting UV optical fibers with other sizes may be selected depending on the amount of energy to be delivered. The side-emitting UV optical fibers are generally able to deliver UV wavelengths for up to about 0.1 mm in the surrounding region, i.e. provide a wavelength penetration depth of about 0.1 mm or lower, such as 0.09 mm or lower, with sufficient energy to denature circulating tumor cells that are attached to the device.

Optionally, one or more portions of the UV emission region are coated to prevent UV emission. In those regions where side-emission of energy is not desired, an opaque coating may be provided or treatment such as by sand-blasting may be provided to scatter wavelength back into the axial direction of the fibers.

### Magnetic Particles

The shaft region also includes a plurality of magnetic nanoparticles or microparticles (referred to herein as “magnetic particles”) 310, which are attached to the outer surface of the shaft region. The magnetic particles can have the same or different diameters. Suitable diameter ranges include 1 mm to 10 mm, or 0.1 mm to 1 mm. Optionally the magnetic particles 1 micron or less in size. The magnetic particles can be arranged end-to-end along the surface of the shaft region of the device, with alternative magnetic polarities.

### Non-magnetic Spacers And/or Fins

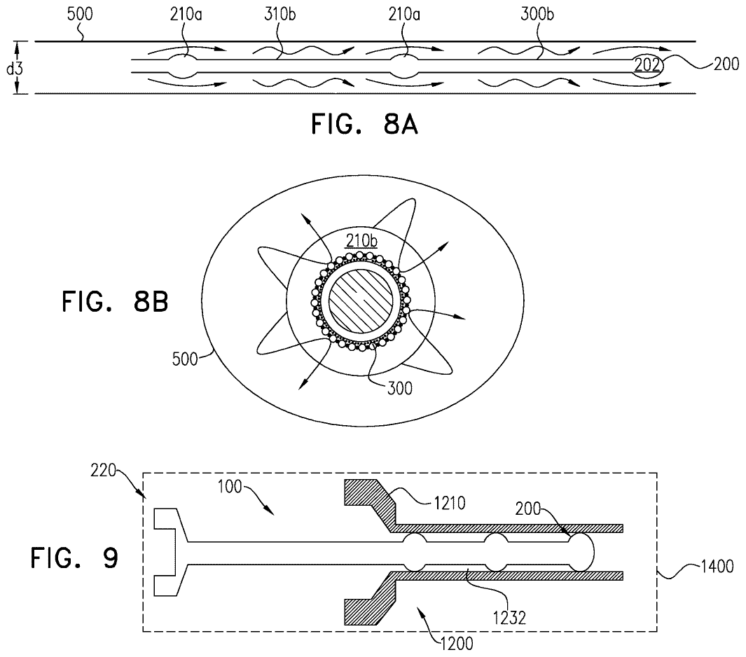

In some applications, device 100 also includes one or more, typically at least two, optionally three or more non-energy emission regions which do not emit UV energy. Non-energy emission regions optionally include one or more spacers 210 (shown in FIG. 8A, for example). The spacers can be located between different shaft regions, such as between a first shaft region and a second shaft region.

To prevent the device from contacting the blood vessel walls, one or more spacers may be periodically positioned along the length of the device. Spacers may be formed from a non-magnetic material, such as a metal such as a non-ferrous metal, such as stainless steel, or a polymeric material. Optionally, the spacer is a hemocompatible, synthetic material, such as polyethylene oxide (PEO), phosphorylcholine (PC), or poly 2-methoxyethylacrylate.

The spacers have diameters that are greater than the diameter of the wire, such as diameters that are at least 10% greater, at least 20% greater, at least 30% greater, at least 40% greater, at least 50% greater, at least 60% greater, such as between about 50% and 70% greater, of between about 60% and 70% greater, or larger, optionally the diameter of the spacers is 1.5-3 times greater, such as 1.5-2 times greater, than the diameter of the wire, are located in one or more positions along the length of the device to slow down the blood flow. The diameter of the spacers can be the same or different from each other and/or can be the same or different from the diameter of the end protector at the insertion end of the device.

However, the diameter of the spacer is small enough to fit inside and to be injected through the lumen of a syringe needle to facilitate implantation in the subject. For example, if the lumen of the syringe needle is about 1.8 mm in diameter the spacer and/or end protector has a diameter of less than 1.8 mm, such as 1.7 mm or smaller, 1.6 mm or smaller, or 1.5 mm or smaller.

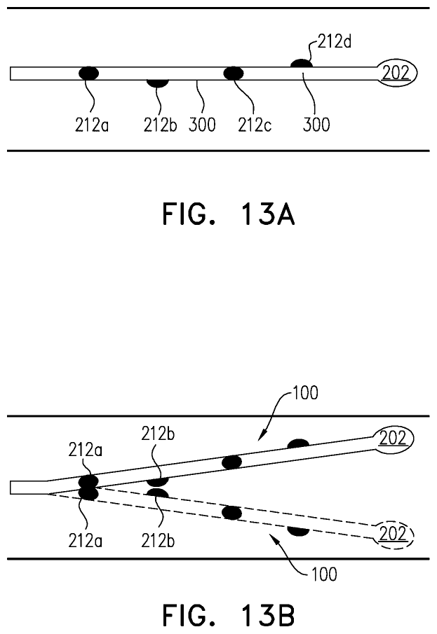

In some applications, the device includes two or more fins 212 (shown in FIG. 13A, for example) protruding from different locations along the outer surface of the shaft region. The locations of the two or more fins are selected to provide flexing forces along the length of the device when the device is inserted in a blood vessel and subjected to blood flow. The fins increase turbulence in the blood flow compared to the same device where the shaft region that does not contain any fins. The fins can have any suitable shape or size. For example, the fins can be in the shape of hemispheres, where the substantially flat side is attached to the surface of the tubular core and the curved surface protrudes therefrom. Optionally a first fin is located in a first location on the outside of the tubular core and/or shaft region, a second is located a set length away from the first location and a set number of degrees rotation from the first location, such as about 10°-90°, optionally from about 30°-90°, from about 30°-90°, from about 30°-45°, from about 30°-60°, from about 60°-90°, from about 70°-90°, or from about 80°-90°, from the first location. Similarly, additional fins can be located the same linear and rotational distances from the adjacent fin. For example, a third fin can be placed at a similar linear and rotational distance from the second fin as the second fin is from the first fin. One or more additional fins can be located in a similar manner along the length of the saft region of the device. In place of the same linear and rotational distances between a first fin and the adjacent fin, different linear and rotation distances can be selected between two or more fins on the outside of the tubular core and/or shaft region of the device.

Following implantation, when the device is subjected to a subject's blood flow, the blood flow current exerts a pressure against the end protector at the insertion end of the device and continuously creates a random flexing motion long the length of the device in all directions. The movement of the device when subjected to the blood flow allows a greater volume of blood to contact the device than if the same device was stationary in the blood vessel.

### Insertion End of the Device

The insertion end 200 of device 100 (shown in FIG. 9 for example) is a non-energy emitting region.

The end protector 202 located at the insertion end of the device typically has a diameter greater than the diameter of the wire, such as at least 40% greater, at least 50% greater, at least 60% greater, such as between about 50% and 70% greater, of between about 60% and 70% greater, or larger, optionally the diameter of the end protector is 1.5-3 times greater, such as 1.5-2 times greater, than the diameter of the wire. The insertion end is configured to facilitate insertion into the blood vessel without puncturing the wall of the blood vessel. Additionally, the insertion end typically has a suitable geometry to prevent the blood vessel walls from contacting the shaft region of the device, and thereby prevent the UV energy from damaging the walls of the blood vessel.

Optionally, the insertion end is formed of a hemocompatible, non-magnetic material, or a material with low levels of magnetism, such as a stainless steel. Alternatively, the insertion end can be formed of another hemocompatible, non-magnetic material, such as polyethylene oxide (PEO), phosphorylcholine (PC), or poly 2-methoxyethylacrylate.

The end protector 202 at the insertion end can have any suitable geometry that is configured to facilitate insertion into the blood vessel without puncturing the wall of the blood vessel. Additionally, end protector has a suitable geometry to prevent the blood vessel walls from contacting the shaft region of the device. For example, the insertion end can be in the shape of a sphere, ovoid, ellipsoid, lima bean-shaped, mushroom cap-shaped, etc.

When the end protector has a non-spherical geometry with two or more diameters, its greatest diameter is as at least 10% greater, at least 20% greater, at least 30% greater, at least 40% greater, at least 50% greater, or at least 60% greater than the diameter of the wire, such as between about 50% and 70% greater, between about 60% and 70% greater, than the diameter of the wire, optionally the largest diameter of the end protector is 1.5-3 times greater, such as 1.5-2 times greater, than the diameter of the wire.

### Attachment End of the Device

The attachment end 220 of device 100 (shown in FIG. 9, for example) is located opposite the insertion end and typically remains outside of the body when the device is in use. Typically the attachment end of the device is a non-energy emitting region.

The attachment end is typically formed from a non-magnetic material, which can be the same or different than the material for the spacers and/or the end protector.

The attachment end of the device contains an attachment region configured to attach to a syringe piston or another insertion element to push the device into the desired position in the subject's body. The attachment end can include one or more depressed regions configured to mate with a syringe plunger, which can include a tip that is configured to mate with the one or more depressions in the attachment region.

Optionally, the attachment end is configured to attach to and be removable from a first connector, which connects with the end of the plunger. Optionally, a second connector is provided to attach the attachment end to the external UV generator. The first and/or second connectors may be provided as part of a system for using the device.

### System for Implantation and Use of the Implantable Device

A system for implanting device 100 in a small blood vessel in a subject typically includes an insertion device, such as a syringe, and the device described above. The insertion device can be a syringe containing a needle, a barrel and a plunger.

The implantable device can be provided in a sterile, sealed container, such as a capsule or other packaging. The implantable device can be prepackaged in a sterilized capsule or other container or packaging.

Optionally, the sterile capsule also contains a syringe needle. In these applications, the implantable device may be located inside the syringe needle to facilitate insertion and prevent cross-contamination. The device may be pre-loaded in the needle, such that the insertion end is located inside the needle lumen.

Optionally the syringe plunger is provided in the same packaging or in a separate container.

Optionally the sterile container includes a needle, the syringe barrel and the implantable device. In this embodiment, the needle hub is attached to the syringe barrel. When assembled in the container, substantially all of the implantable device can be located inside the syringe barrel and needle.

Optionally the sterile container includes a needle, the syringe barrel, the plunger, and the implantable device. Optionally, the plunger is attached to the attachment end of the device. Alternatively, the plunger can be separated from the implantable device and inserted into the barrel at the time of use and depressed to contact and push the attachment end of the device into the desired site during insertion.

The syringe typically has an opening with a suitable configuration and dimensions to attach to the needle hub and allow for the needle hub to be detached following insertion of the needle and device into the subject.

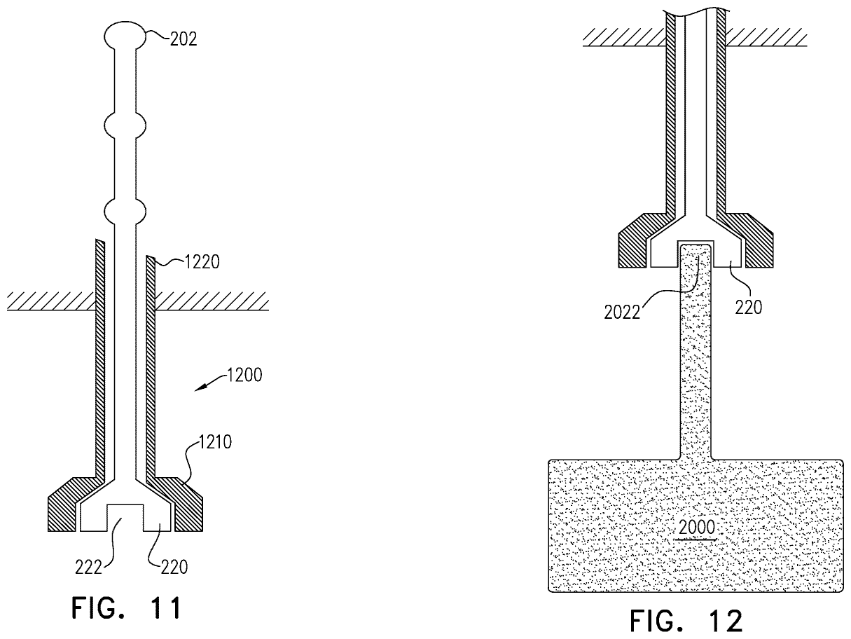

Optionally, an external UV generator 2000 (shown in FIG. 12) is provided with the system for implantation of the device. Optionally, an external UV generator is provided separately from the system for implantation of the device. The UV generator may include or be in electrical communication with a timer, to turn the UV generator on and off to deliver the UV rays to the subject for a set period of time. The particular UV wavelengths to be delivered can be selected on the UV generator, as well. The UV generator may include one or more connection ports to attach to a connector at a first end, which is configured to attach to the attachment end of the implantable device at a second end.

### Methods of Using the Implantable Devices

Device 100 is injected subcutaneously using a suitable insertion device, such as a syringe or other insertion device, into a small blood vessel. The insertion site is disinfected prior to insertion of the device.

Typically, prior to insertion, the insertion end of the device is located inside the syringe needle.

An insertion device, such as a needle initiates the entrance of the device into a small blood vessel. The plunger of the insertion device, such as a syringe, is depressed to push the device into the desired site. In some applications, following insertion, the insertion end of the device is located about 1-5 mm, 1-2 mm, 1-3 mm, or 1-4 mm below the surface of the subject's skin. This insertion depth is particularly useful for shorter devices, which can be inserted in small blood vessels in a variety of locations, such as in the subject's wrist and lower arm. In other applications, following insertion, the insertion end of the device is located about 5 -15 cm, such as 5-11 cm, 5-10 cm, 6-10 cm, 6-12 cm, 8-12 cm, or 8-10 cm, beneath the surface of the subject's skin. This insertion depth is particularly useful for longer devices, which can be inserted in blood vessels in a variety of locations, such as in the subject's leg, for example in the femoral vein.

Following insertion, the piston is separated from the attachment mechanism, and the attachment mechanism is outside the subject's body.

The site is disinfected, as needed. Optionally a sterile bandage is placed around the insertion site to prevent infection in and around the insertion site.

Typically, the shaft of the needle remains inside the insertion site, while the needle hub typically remains outside of the insertion site, while the device is implanted in the subject. The attachment mechanism of the device is typically inside the needle hub, and located above the outer surface of the subject's skin at the site of the injection.

Optionally, a saline irrigation port is provided to clean and disinfect the interface between the device and the skin at scheduled interval times. The device may be implanted in a subject for days, weeks, or a month.

When UV eradication of CTC bound on device is to be performed, the attachment end is attached directly or indirectly with UV generator 2000 (shown in FIG. 12). While the UV generator can be used an any time of the day, in some instances subjects may choose to attach to the UV generator when they are resting, such as in the evening. The UV generator runs for as long as needed in each session, such as for up to 1 minute, 1-10 minutes, up to 30 minutes, up to 1 hour, or 1-5 hours, or any suitable period of time.

The device typically remains in the desired site for at least a few days, optionally for at least one week, optionally for two weeks, or even longer. After this time period, the device is removed and typically, a second device is inserted in the same site or a different site in the subject. Use of different blood vessel insertion sites can help prevent chronic inflammation in the subject. Optionally, following removal, the previously implanted device is cleaned sterilized and recycled for future use.

A device is typically inserted into the subject for a suitable period of time (such as 1-5 days, 1 week, 2 weeks or longer), used with UV generator 2000 (shown in FIG. 12), as described above, and removed. These steps can be repeated for 1 month, 2 months, or longer, such as 6 months, or 1 year, or even longer, until no CTC are detected in the subject's blood stream.

The use of the device described herein can begin as soon as possible following a subject's diagnosis with a specific cancer. The biomarkers are selected to bind to the specific cancer.

Optionally, prior to surgery to remove a primary tumor, the device described herein is inserted into a small blood vessel of the subject to remove any CTC that may be shed during the surgery.

Optionally more than one device, such as 2, 3, 4, 5 or more devices, are implanted in the subject in different small blood vessels and remains in the subject at the same time. In use, when multiple devices are implanted in the subject at the same time, typically more than one of the devices, optionally all of the implanted devices, will be attached to the UV generator at the same time to increase the likelihood of coupling the circulating CTC to the biomarkers on the particles.

Due to the magnetic attraction between the nanoparticles and the wire, the nanoparticles remain attached to the wire at the site of implantation. Thus, the nanoparticles do not travel through the blood stream and are not systemically delivered.

In some applications, following capture, the CTC are exposed to ultraviolet light for an effective period of time to denature the cells in situ. Typically, the CTC are exposed to UV light for a short period of time, such as 10-30 seconds, or 10 second to 1 minute. The implantable capture device is injected subcutaneously and is implanted in a small blood vessel or a large blood vessel, depending on the needs of the subject. For example, when implanted in a small blood vessel, the device is located a few millimeters beneath the outer surface of the subject's skin. In such applications, the device has a length of about 2 to 3 cm and when inserted the insertion end is located about 1 to 5 mm beneath the surface of the subject's skin. In some applications, the device is implanted in a larger blood vessel, such as the femoral vein. In such applications, the device has a length of about 10 cm or longer and when inserted the insertion end is located about 5 cm to 15 cm, such as about 8 to about 10 cm beneath the surface of the subject's skin. Following insertion of the longer implantable devices, the shaft region and insertion end of the device flex and twist when subjected to the forces in the blood flow.

The device typically contains central tubular magnetic, flexible core 340, such as in the form of a wire or filament. Additionally, magnetic particles, which may contain or are formed from one or more ferromagnetic materials and are attached to one or more biomarkers, are attached to the outer surface of the wire. The nanoparticles are typically in contact with the wire via magnetic forces. In some applications, the wire or filament is surrounded by side-illumination emitting optical fibers that are able to transmit ultraviolet (UV) light along their length. The optical fibers can contain a hemocompatible, non-thrombogenic outer layer or be coated with an anti-coagulant, such as heparin.

The insertion end of the device typically contains an inert, non-magnetic end protector that has a diameter which is greater than the diameter of the wire, which can be in the shape of a curved tip, such as in the form of a ball or other curved shape, such as a lima bean shape. Optionally, spacers having diameters greater than the diameter of the diameter of the wire are located in one or more positions along the length of the wire to create turbulence in the blood flow and/or slow down the blood flow.

The opposite end of the device typically contains an attachment region configured to attach to a piston to push the device into the desired position in the subject's body. Following insertion, the piston is separated from the attachment region, and the attachment region is located outside the subject's body. The attachment region is also configured to attach to and be removable from external UV generator 2000 (e.g., as shown in FIG. 12). Such attachment can be direct or indirect, such as via an additional attachment device.

In use, following insertion, the device remains in situ for a sufficient period of time to attract and bind CTC to the surface of the device. In some applications, the device is attached to a UV generator, the device delivers UV radiation when desired or at preset intervals of time to the subject, in a sufficient amount to denature any CTC that are attached to the device. Suitable intervals include 8 to 10 hours following insertion, optionally every day, such as every evening, the device is attached to a UV generator for a suitable time period, such as for about 1 minute or less, optionally for about 5-10 minutes or less.

Due to the magnetic attraction between the nanoparticles and the wire, the nanoparticles remain attached to the wire at the site of implantation. Thus, the nanoparticles do not travel through the blood stream and are not systemically delivered.

The device typically remains in the desired site for at least a few days, optionally for at least one week, optionally for two weeks, or even longer. Afterwards, the device is removed. A new device is inserted or implanted at the same or a different site in the subject. Optionally, following removal, the device is cleaned, sterilized and prepared for reuse. Optionally following removal and cleaning, the initial device is reinserted at the same or a different site beneath the surface of the subject's skin, for as long as needed.

Portions of exemplary implantable devices 100 are depicted in FIGS. 6A, 6B, 7A-C, 8A, and 8B. As shown in FIGS. 6A, 6B, and 8A, device 100 contains one or more shaft regions 300 (with respective regions labelled 300a, 300b, etc.) which are formed of a central tubular magnetic, flexible core 340, such a wire or filament, which is surrounded by optical fibers 330 that are able to transmit ultraviolet (UV) light. The outer surface of the optical fibers is formed of a non-thrombogenic material or is coated with a non-thrombogenic coating and/or an anti-coagulant 320, such as heparin. Additionally, magnetic particles 310 (with respective particles labelled 310a, 310b, etc.) such as microparticles or nanoparticles, that are attached to one or more biomarkers 312 (with respective biomarkers labelled 312a, 312b, etc. in FIGS. 7B-C) are attached to the outer surface of the wire. The magnetic particles 310a, 310b, are typically formed from or contain one or more magnetic materials, such as iron.

The device also contains an end protector 202 at the insertion end 200, an attachment end 220, and optionally one or more spacers 210 (with respective spacers labelled 210a, 210b, etc. in FIG. 8A). In some applications, such as illustrated in FIGS. 13A and 13B, the shaft region of the device includes two or more fins 212 (with respective fins labelled 212a, 212b, etc.) such as a plurality of fins, located in a variety locations along the length of the shaft region 300 device. The fins 212 can have any suitable geometry to induce movement along the length of the shaft region when the device is in contact with blood flow. For example, as shown in FIG. 13A, the fin 212 can be in the form of a hemisphere, with a substantially flat attachment end 211 and a rounded outer surface 213.

FIGS. 13A and 13B depict a device 100 that contains four fins attached to the outer surface of the shaft region 300, in accordance with some applications of the present invention. However, fewer or more fins can be attached to the outer surface of the shaft region. Additionally, the fins may be attached in a repeating pattern or randomly. The exemplary device depicted in FIGS. 13A and 13B contains a repeating pattern for the location of the fins, where the first fin 212a is located in a first location on the outer surface of the shaft region, the second fin 212b is located a set length away from the first location and a set number of degrees from the first location, such as about 90° from the first location. The third fin 212c is located the same linear and rotational distance from the second fin 212b as the linear and rotational distance of the first fin from the second fin. Similarly, the fourth fin 212d is located the same linear and rotational distance from the third fin 212c as the first fin is from the second fin.

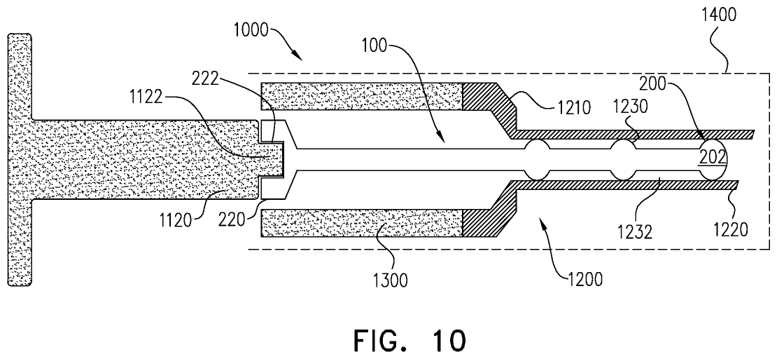

The device depicted in FIGS. 13A and 13B can be modified to include additional fins that are aligned in a similar manner along the length of the saft region 300 of the device. Optionally, the device depicted in FIGS. 13A and 13B can be modified such that in place of the same linear and rotational distances between two adjacent fins in the plurality of fins located on the surface of the tubular core and/or shaft region, different linear and rotation distances can be selected between two or more fins on the outside of the tubular core and/or shaft region of the device. As shown in the figures, such as FIGS. 10, 13A, 13B, 14A-14C, 15A, and 15B, the insertion end 200 of device 100 typically contains a non-magnetic end protector 202, which has a greater diameter than the diameter of the diameter of the core with optical fiber surrounding it. The end protector can have any suitable shape that has a diameter that is greater than the diameter of the energy emission regions and terminates in a rounded surface. Suitable shapes include spheres, lima bean-shaped, mushroom cap-shaped, ovoids, ellipsoids, etc. Optionally, one or more spacers 210a, 210b, each of which has a diameter greater than the diameter of the diameter of the core with optical fiber surrounding it are also located in one or more positions along the length of the core.

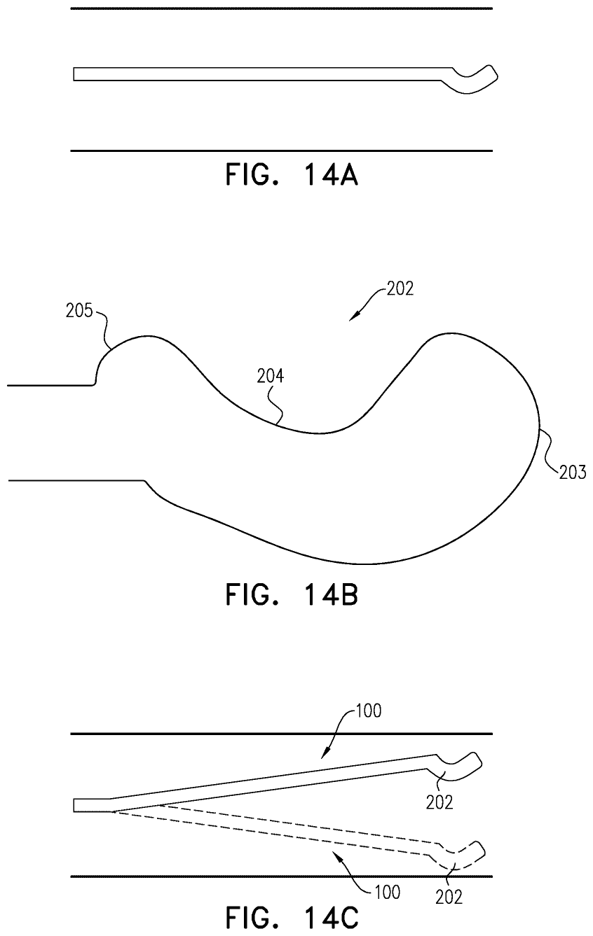

As shown in FIGS. 10, 13A, 13B, 15A, and 15B, end protector 202 can be in the shape of a sphere. As shown in FIGS. 14A-14C, the end protector 202 can be in other curved shapes, such as the shape of a lima bean. As shown in FIG. 14B, the end protector 202 can terminate with a rounded end 203 at the insertion end of the device. Adjacent to the insertion end, the end protector can include a central non-symmetrical concave portion 204, followed by a convex protrusion 205 where the end connector attaches to the shaft region 300.

The attachment end 220 of the device contains an attachment region configured to attach to a syringe piston or another insertion device 1000 to push the device into the desired position in the subject's body. As shown in FIGS. 10 and 11, the attachment end 220 can include one or more depressed regions 222 configured to mate with the distal end 1120 of a syringe plunger 1100. The distal end 1120 of the plunger can include a corresponding tip 1122 that is configured to mate with the one or more depressions in the attachment region.

Optionally, the attachment end 220 is configured to attach to a separate connector (not shown), which connects with the end of the plunger. Optionally a second connector (not shown) may be provided to attach the attachment end to the external UV generator.

Following insertion, the piston is separated from the attachment mechanism, and the attachment end is outside the subject's body. The attachment end 220 is also configured to attach to and be removable from an external UV generator 2000 via a connector 2022, as shown in FIG. 12.

FIGS. 10 and 11 depict systems for injecting an exemplary implantable device 100 in a subject's blood vessel, such as a small blood vessel in the wrist or a larger blood vessel, such as the femoral vein.

The insertion device 1000 typically includes a needle 1200, barrel 1300, and a plunger 1100.

The insertion device, optionally just the needle, and the implantable device, are provided in a sterile package or container 1400 (shown in FIGS. 9 and 10). The sterile package can be opened at the back end to facilitate assembly of the insertion device, such as attachment of the plunger to the attachment end of the device. Even when the package is opened, the needle tip should remain covered and inside the sterile package or container until the time of use.

The needle 1200 typically includes a needle hub 1210 and a needle shaft 1230, which terminates at its insertion end with a needle tip 1220. The center of the shaft is hollow and contains a lumen 1232, through which the device is inserted into the subject.

The sterile package 1400 may also contain the needle for insertion. Optionally the plunger is provided in the same package or in a different package. As shown in FIG. 9, the device may be pre-loaded in the needle, such that the insertion end is located inside the needle lumen 1232. The needle, with the shaft region of the device included in the lumen of the needle, is enclosed in a sealed sterile capsule or package 1400.

Optionally the needle hub 1210 is attached to the syringe barrel 1300. In this embodiment, substantially all of the implantable device is inside the syringe barrel and needle. Optionally, the plunger is attached to the attachment end of the device. As shown in FIG. 5, the plunger has a tip 1122 configured to fit inside and mate with the depressed region 222 of the attachment end 220 of the device 100.

Alternatively, the plunger can be inserted into the barrel at the time of use and depressed to contact and push the attachment end of the device into the desired site during insertion.

### Exemplary Methods for Using the Implantable Device.

Prior to insertion, the insertion site is typically cleaned and disinfected. Prior to insertion, the insertion end 200 of the device is inside the needle lumen 1232 and the plunger 1100 is attached to or in contact with the attachment end 220 of the device. As shown in FIG. 10, the plunger's tip 1122 is in contact with and mates with the depressed region 222 of the attachment end 220 of the device 100.

During insertion, the tip of the needle is inserted into the insertion site and the plunger of the insertion device, such as a syringe, is depressed to push the device into the desired site. In some applications following insertion, the insertion end of the device is located a few millimeters, such as 1-5 mm, 1-2 mm, 1-3 mm, or 1-4 mm, below the outer surface of the subject's skin. In other applications following insertion, the insertion end of the device is located about 5-15 cm, such as 5-11 cm, 5-10 cm, 6-10 cm, 6-12 cm, 8-12 cm, or 8-10 cm below the outer surface of the subject's skin.

Following insertion, the plunger is removed and separated from the attachment end 220, and the syringe barrel 1300 is removed and separated from the needle hub 1210. The attachment end 220 is inside the needle hub 1210, and both of the attachment end 220 and the needle hub 1210 are outside of the subject's body, adjacent to the outer surface of the skin at the insertion site. Optionally a portion of the needle 1200 is also located outside of the subject's body, while the needle tip 1122 remains beneath the skin surface. The needle is typically secured to the skin in the desired location to prevent accidental removal or movement.

Optionally a sterile bandage is placed around the insertion site to prevent infection in and around the insertion site.

Schematics showing an exemplary implantable device inside a blood vessel and CTC 400 (with respective CTC being labelled 400a, 400b, etc.) circulating in the blood flow surrounding the device are provided in FIGS. 7A, 7B, 8A, and 8B.

Following implantation, the device 100 remains at the site of implantation and is located inside the small blood vessel 500, away from the walls of the blood vessel. This allows the blood to flow around the device. As CTC 400a, 400b flow near the device, they are attracted to the device by the biomarkers 312a, 312b and bind to the biomarkers, which are bound to the magnetic particles 310a, 310b.

As depicted in FIG. 7C (which shows a slice of implantable device 100), the tubular magnetic core 340 is in the center, and is surrounded by one or more side-emitting UV optical fibers 330, which can be coated with an anticoagulant, such as coagulant 320, magnetic particles 310a, such as nanoparticle or microparticles, on which biomarkers 312a are attached are connected to the device via magnetic attraction to the core 340. The biomarkers are selected to bind to the specific cancer that has been detected in the subject.

In some applications, one or more spacers 210a, 210b are included along the length of the device, such as illustrated in FIGS. 8A and 8B, and create a fluctuating blood current that also slows down the blood flow as it passes the spacers. This can cause the CTC to bounce onto the surface of the implanted device thereby facilitating the coupling of the CTC to the biomarkers. If the blood flow is slowed down, the CTC can remain near the biomarkers for a longer time period, thereby facilitating their coupling to the biomarkers. The curved lines with arrows and squiggly lines with arrows depict a hypothetical, exemplary blood flow, which bounces up and down along the length of the implanted device inside the blood vessel. The insertion end 200 and the spacers 210a, 210b have rounded outer surfaces and greater diameters than first and second shaft regions 300a, 300b. Thus, the end protector at the insertion end and the spacers, if present, protect the blood vessel wall surface by preventing puncturing and rubbing and also prevent the UV rays from emitting too close to the walls of the blood vessel. In some applications, one or more spacers 210a, 210b are included along the length of the device, such as illustrated in FIGS. 8A and 8B, and create a fluctuating blood current that also slows down the blood flow as it passes the spacers. This can cause the CTC to bounce onto the surface of the implanted device thereby facilitating the coupling of the CTC to the biomarkers. If the velocity of blood flow is decreased, the CTC remain near the biomarkers for a longer time period, thereby facilitating their coupling to the biomarkers. The curved lines with arrows and squiggly lines with arrows depict a hypothetical, exemplary blood flow, which bounces up and down along the length of the implanted device inside the blood vessel. The insertion end 200 and the spacers 210a, 210b have rounded outer surfaces and greater diameters than first and second shaft regions 300a, 300b. Thus, the end protector at the insertion end and the spacers, if present, protect the blood vessel wall surface by preventing puncturing and rubbing and also prevent the UV rays from emitting too close to the walls of the blood vessel. In some applications, such as shown in FIGS. 13A and 13B, the shaft region of the device includes a plurality of fins. As the blood flow contacts the fins 212a, 212b, etc., it pushes the insertion end and the shaft region of the device in a random pattern, including side to side movements. This is depicted in FIG. 13B, which shows the device moving from one side of the blood vessel (with the device indicated by solid lines) to the other side of the vessel (with the device indicated by dashed lines). Thus, when the implant is in contact with the blood flow, a random flexing motion that runs along the length of the shaft region in all directions occurs continuously. The concave and convex shaping of opposing sides of the shaft region creates flexing sideways forces, rebounding due to tensile strength of the core. The movement of the device allows a greater volume of blood to be in contact with the surface of the device during a given time period compared to the same device if it was stationary. This increases the likelihood of coupling between the biomarkers and the CTC.

The end protector of the device can have any suitable geometry that protects the walls of the blood vessel. Optionally, the end protector has a suitable geometrical configuration to facilitate movement of the device when it is subjected to the blood flow. For example, the end protector can be lima bean shaped. For some applications, the end protector 202 is pushed from side to side, causing the implanted device 100 to flex from side to side and in a random flexing motion when subjected to the flow of blood. This is depicted in FIG. 14C, which shows the device moving from one side of the blood vessel (indicated with solid lines) to the other side of the vessel (indicated with dashed lines).

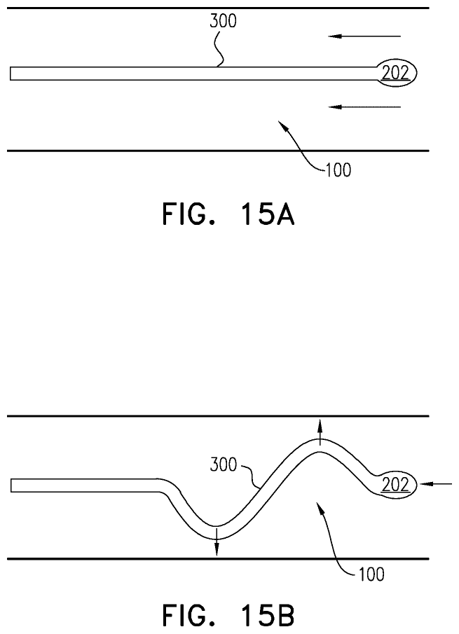

Any of the exemplary devices described herein can be inserted into a subject's blood vessel in a direction that opposes the direction of the blood flow. As shown in FIG. 15A, the blood flow is direction upstream of the insertion end of the device. As shown in FIG. 15B, the pressure of the blood flow pushes against the end protector and contracts the length of the shaft region 300, thereby twisting the shaft region along its length, resulting in the shaft region having a spring-like twisting configuration.

Optionally, at preset time periods or when desired, the subject or another individual such as a healthcare provider or assistant, connects the attachment end 220 to UV generator 2000 via connector 2022, as shown in FIG. 12.

While the UV generator can be used an any time of the day, in some instances subjects may choose to attach to the UV generator when they are resting, such as in the evening. The UV generator is used for as long as needed in each session, such as for up to 1 minute, for 1-10 minutes, 30 minutes, 1 hour, or 1-5 hours, or any suitable period of time to denature the CTC that are attached to the device at the time that the UV rays are emitted. For example, the attachment end may be attached to a UV generator at night before a subject goes to bed for a short period of time, such as about one minute, less than one minute, less than 2 minutes, 1-10 minutes, or 1-30 minutes, to denature CTC that are attached to the device at that time.

Device 100 can be removed after about 1 day, 2 days, 2-5 days, 1 week, 2 weeks, 3 weeks, or longer, following insertion and a new device can be inserted in the same location or in a different insertion site in the subject. The process described above is then repeated for the new device and additional new devices can be similarly inserted, as needed, until no additional CTC are observed in the subject's blood.

There follow a description of components of implants and implantable devices (e.g., magnetic particles, functional groups, biomarkers, antigens, ligands and receptors, antibodies, Fc domains, and anti-coagulant materials) that are applicable both to implants that are generally as described hereinabove with reference to FIG. 1A-5, as well as to implantable devices that are generally as described hereinbelow with reference to FIG. 6A-15B.

### Implantable Devices

Implants and implantable devices described herein can typically be easily delivered via injection, such as via subcutaneous injection or intravenous injection, and remain in the subject for a prolonged period of time are described herein. While in the subject, CTC that flow past the wire can attach to the one or more biomarkers that are on the outer surface of the device. In some applications described herein, the one or more biomarkers are manipulated to be released from the implantable device, to capture CTC in the blood stream, and to return to the implantable device, as will be described in further detail hereinbelow.

### Magnetic Particles

In some applications of the present invention, portions of the implantable device include a plurality of magnetic nanoparticles or microparticles (referred to herein as “magnetic particles” or “magnetic beads”), which are attached to regions in the implantable devices. Additionally, or alternatively, magnetic particles are antibody (or another biomarker)-modified magnetic particles by having an antibody conjugated thereto. The magnetic particles can have the same or different diameters.

### Sizes for Magnetic Particles

Suitable diameter ranges for the magnetic particles include 1 mm to 10 mm, or 0.1 mm to 1 mm. Optionally the magnetic particles 1 micron or less in size. The magnetic particles can be microparticles with a mean particle size of less than about 1000 microns. In some forms, the microparticle has a size of between about 100 μm and about 500 μm, between about 100 μm and about 400 μm, between about 100 μm and about 300 μm, between about 100 μm and about 200 μm, between at least 1 μm and about 100 μm, between at least 1 μm and about 50 μm, or between at least 1 μm and about 10 μm. In some forms, the microparticle has a size of between at least 10 nm and about 1 μm.

The magnetic particles can be nanoparticles with a mean particle size of less than one micron. In some forms, the nanoparticle has a size of between at least 50 nm and less than 1 μm, between at least 100 nm and less than 1 μm, between at least 200 nm and less than 1 μm, between at least 300 nm and less than 1 μm, between at least 400 nm and less than 1 μm, between at least 500 nm and less than 1 μm, or between at least 600 nm and less than 1 μm. In certain forms, the nanoparticles have a mean particle size of about 500 nm, 200 nm, 100 nm, or 50 nm, or 10 nm.

Mean particle size generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. The diameter of a non-spherical particle may refer to the largest linear distance between two points on the surface of the particle. Mean particle size can be measured using methods known in the art, such as dynamic light scattering.

### Materials for Magnetic Particles

The magnetic particles can be formed from any biocompatible, magnetic material with a sufficient magnetic field strength to remain attached to the implantable device while the device is in the subject's body. The magnetic materials are typically permanent magnets. Suitable materials for the magnetic particles include but are not limited to ferromagnetic materials, such as neodymium, iron, nickel, cobalt, and gandolinium, and samarium, and magnetic alloys thereof. In some applications, the particles are iron microparticles or nanoparticles.

### Functionalization

The particles can include reactive functional groups that can be utilized for functionalization with biomarkers, therapeutic, prophylactic and/or diagnostic agents. These may be small molecule active agents or biomacromolecules, such as proteins, polypeptides, or nucleic acids. Suitable small molecule active agents include organic and organometallic compounds. The small molecule active agents can be a hydrophilic, hydrophobic, or amphiphilic compound. It may also be advantageous to incorporate onto or into the particle, a contrast agent, radiopaque markers, fluorescent dye, or other additives to allow the particles to be imaged in vivo for tracking, positioning, and other purposes.

Exemplary reactive functional groups include, but are not limited to, carboxylic acid and activated derivatives thereof, amino, maleimide, thiol, sulfonic acid and derivatives thereof, carbonate and derivatives thereof, carbamate and derivatives thereof, hydroxyl, aldehyde, ketone, hydrazine, isocyanate, isothiocyanate, phosphoric acid and derivatives, phosphonic acid and derivatives, haloacetyl, alkyl halides, vinyl sulfone, vinyl ketone, epoxide, oxirane, and aziridine.

In some applications, the particles are coated to provide attachment site for functional moieties. For example, the surface of the particle can be modified through the creation of a few atomic layers of organic (polymer) or inorganic (metal or oxide) surfaces.

In general, the polymer or polymers are selected based on desired properties and the application in which it is going to be used. The polymeric matrix may be formed from non-biodegradable or biodegradable polymers; however, preferably, the polymeric matrix is biodegradable. The polymeric matrix can be selected to degrade over a time period ranging from one day to one year. In general, synthetic polymers are preferred, although natural polymers may be used.

### Biomarkers

In some applications, magnetic particles are functionalized with a biomarker. Typically, the biomarker is selected to facilitate attraction of the CTC to the particles.

The term “biomarker” is broadly applied to any material that can facilitate binding to the circulating tumor cells. The biomarkers may be targeting moieties, molecules, cells, genes, gene products, enzymes, drugs, hormones or other materials that include one or more portions that are attracted to CTC. Examples of biomarkers that may be used to coat the particles include, but are not limited to ligands for EpCAM, CK8, CK18, CK19, E-cadherin, ZO 1, ESPR 1, HER 2.

Additionally, or alternatively, other materials that promote binding of the particle to the CTC of interest may be attached to the particle. The biomarkers can be antigen-binding targeting domains. In some applications, the targeting domains bind to antigens, ligands or receptors that are specific to tumor cells, or are upregulated in tumor cells compared to normal tissue. In some applications, the targeting domains bind to antigens, ligands or receptors that are specific to immune tissue involved in the regulation of T cell activation in response to infectious disease-causing agents. Tumor-Specific and Tumor-Associated Antigens

In some applications, the biomarker specifically binds to an antigen that is expressed by tumor cells. The antigen expressed by the tumor may be specific to the tumor or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are contemplated for use in certain applications.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melanoma associated antigens, etc.).

Genes that encode cellular tumor associated antigens include cellular oncogenes and proto-oncogenes that are aberrantly expressed. In general, cellular oncogenes encode products that are directly relevant to the transformation of the cell, and because of this, these antigens are particularly preferred targets for immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

Additional tumor antigens that can be targeted, including a tumor-associated or tumor-specific antigen, include, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS- 9, pml-RAR alpha fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, beta-Catenin, CDK 4, Mum-1, p16, TAGE, PSMA, PSCA, CT7, telomerase, 43-9F, 5T4, 791Tgp72, alpha-fetoprotein, 13HCG, BCA 225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16,TA-90(Mac-2 binding protein\cyclophilin C-associated protein), TAAL6, TAG72, TLP, and TPS.

In another application, the biomarker specifically binds to a chemokine or a chemokine receptor.

### Ligands and Receptors

In some applications, tumor targeting domains are ligands that bind to cell surface antigens or receptors that are specifically expressed on tumor cells or are overexpressed on tumor cells or as compared to normal tissue. Tumors also secrete a large number of ligands into the tumor microenvironment that affect tumor growth and development. Receptors that bind to ligands secreted by tumors, including, but not limited to growth factors, cytokines and chemokines, including the chemokines provided above can also be targeted. Ligands secreted by tumors can be targeted using soluble fragments of receptors that bind to the secreted ligands. Soluble receptor fragments are fragments polypeptides that may be shed, secreted or otherwise extracted from the producing cells and include the entire extracellular domain, or fragments thereof.

### Antibodies

In some applications, tumor targeting domains are antibodies that bind to cell surface antigens or receptors that are specifically expressed on tumor cells or are overexpressed on tumor cells as compared to normal tissue. The term antibody includes natural or synthetic antibodies that bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that bind the target antigen. An antigen-binding fragment of an antibody includes one or more variable regions of an intact antibody. Examples of antibody fragments include Fab, Fa″, F(a″)2 and Fv fragments; diabodies; linear antibodies; single-chain antibody molecules and multispecific antibodies formed from antibody fragments. For example, the term antigen binding fragment may be used to refer to recombinant single chain Fv fragments (scFv) as well as divalent (di-scFv) and trivalent (tri-scFV) forms thereof.

### FC Domains

In some applications, tumor targeting domains are Fc domains of immunoglobulin heavy chains that bind to Fc receptors expressed on tumor cells. The Fc region as used herein includes the polypeptides containing the constant region of an antibody excluding the first constant region immunoglobulin domain. Thus, Fc refers to the last two constant region immunoglobulin domains of IgA, IgD, and IgG, and the last three constant region immunoglobulin domains of IgE and IgM. In a preferred embodiment, the Fc domain is derived from a human or murine immunoglobulin. In a more preferred embodiment, the Fc domain is derived from human IgG1 or murine IgG2a including the CH2 and CH3 regions.

### Anti-Coagulant Material or Coating

If needed to limit or prevent accumulation of blood components on the device, an anti-coagulant coating is provided on the outer surface of the one or more side-emitting optical fibers. The anti-coagulant coating prevents or reduces platelet adhesion and/or activation while the device is in the subject's body and also allows the ultraviolet energy to pass through the coating.

Suitable anti-coagulants that can be coated onto the surface of the device and/or the surface of the side-emitting optical fibers include but are not limited to heparin. For example, the heparin coating can include a coating formed of covalently immobilized heparin on the surface of the implantable device (such as Carmeda Bioactive Surface® (Carmeda, Switzerland) Rheoparin (Medos) and Bioline (Maquet)) or ionically linked heparin (such as Duraflo (Baxter)).

Optionally the surface of the device, the shaft region and/or one or more side-emitting UV optical fibers is coated with a non-thrombogenic coating, such as polyethylene oxide (PEO), phosphorylcholine (PC), poly 2-methoxyethylacrylate, and triblock surface-modifying additives. An example of a commercially available anticoagulant coating includes those hydrophilic medical device coatings sold by HYDROMER and SCS microRESIST parylene coatings.

Although some applications of the present invention are described with respect to antibodies or other biomarkers that are configured to bind with circulating tumor cells, the scope of the present disclosure includes applying the apparatus and method described herein to the removal of microorganisms (such as bacteria or viruses) from the bloodstream, by utilizing antibodies that are configured to bind with such microorganisms, mutatis mutandis.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus for capturing circulating tumor cells (CTC) in vivo, and for use with an intravascular implantable implant body that is implanted in a blood vessel of a subject, the apparatus comprising:
antibody-conjugated magnetic particles configured, upon being released into a bloodstream of the subject, to (a) magnetically attach to the implant body, and (b) selectively bind circulating tumor cells (CTC) in the blood vessel, and a releasing element that is configured to periodically release antibody-conjugated magnetic particles into the subject's bloodstream such as to replenish the implant body with antibody-conjugated magnetic particles.

2. The apparatus according to claim 1, wherein the releasing element comprises a biodegradable material that is degradable into the blood vessel, wherein the antibody-conjugated magnetic particles are embedded in the biodegradable material, and are configured to become released into the blood vessel upon degradation of the biodegradable material.

3. The apparatus according to claim 1, wherein the apparatus is configured for use with an implant body that includes a magnetic coating configured to attract the antibody-conjugated magnetic particles.

4. The apparatus according to claim 1, wherein the antibody-conjugated magnetic particles are configured to attach to the implant following circulation in the blood vessel in response to a magnetic field that redirects that antibody-conjugated magnetic particles to the implant.

5. The apparatus according to claim 1, wherein the antibody-conjugated magnetic particles are configured to selectively bind epCAM protein of the circulating tumor cells (CTC).

6. The apparatus according to claim 1, further comprising an electromagnetic coil unit configured to generate one or more magnetic fields which are applied to the antibody-conjugated magnetic particles.

7. The apparatus according to claim 6, wherein an electromagnetic coil unit is configured to be implanted under skin of the subject.

8. The apparatus according to claim 6, wherein an electromagnetic coil unit is configured to externally coupled to skin of the subject.

9. (canceled)

10. The apparatus according to claim 1, wherein the releasing element comprises an injector configured to inject the antibody-conjugated magnetic particles into the subject's bloodstream.

11. The apparatus according to claim 10, further comprising a computer processor configured to control injection of the antibody-conjugated magnetic particles into the subject's bloodstream at predetermined time intervals.

12. The apparatus according to claim 1, wherein, in response to one or more magnetic fields applied to the antibody-conjugated magnetic particles, the antibody-conjugated particles are configured to detach from the implant body and circulate within a portion of the blood vessel that is under the magnetic field, while being confined to that portion of the blood vessel.

13. The apparatus according to claim 12, wherein the antibody-conjugated magnetic particles are configured to attach to the implant following circulation in the blood vessel in response to termination of the magnetic field.

14-41. (canceled)

42. A method for capturing circulating tumor cells (CTC) in vivo, the method comprising:
implanting an intravascular implantable implant body in a blood vessel of a subject; and
periodically releasing antibody-conjugated magnetic particles into a bloodstream of the subject, the antibody-conjugated magnetic particles being configured, upon being released into the subject's bloodstream, to (a) magnetically attach to the implant body, and (b) selectively bind circulating tumor cells (CTC) in the blood vessel, such as to replenish the implant body with antibody-conjugated magnetic particles.

43. The method according to claim 42, wherein periodically releasing antibody-conjugated magnetic particles into the subject's bloodstream comprises placing a biodegradable material into the blood vessel, wherein the antibody-conjugated magnetic particles are embedded in the biodegradable material, and are configured to become released into the blood vessel upon degradation of the biodegradable material.

44. (canceled)

45. The method according to claim 42, wherein the antibody-conjugated magnetic particles are configured to attach to the implant following circulation in the blood vessel in response to a magnetic field that redirects that antibody-conjugated magnetic particles to the implant.

46. (canceled)

47. The method according to claim 42, wherein, in response to one or more magnetic fields applied to the antibody-conjugated magnetic particles, the antibody-conjugated particles are configured to detach from the implant body and circulate within a portion of the blood vessel that is under the magnetic field, while being confined to that portion of the blood vessel.

48. The method according to claim 47, wherein the antibody-conjugated magnetic particles are configured to attach to the implant following circulation in the blood vessel in response to termination of the magnetic field.

49. The method according to claim 42, wherein periodically releasing antibody-conjugated magnetic particles into the subject's bloodstream comprises periodically releasing antibody-conjugated magnetic particles into the subject's bloodstream using an injector.

50. The method according to claim 49, wherein periodically releasing antibody-conjugated magnetic particles into the subject's bloodstream using an injector comprises using a computer processor to control injection of the antibody-conjugated magnetic particles into the blood vessel of the subject at predetermined time intervals.

51. An implantable medical device for eliminating circulating tumor cells (CTCs) in vivo comprising:
a first shaft region comprising:
a flexible magnetic core;
one or more side-emitting UV optical fibers; and
a plurality of magnetic particles bound to one or more biomarkers, wherein the plurality of magnetic particles are positioned over the optical fibers.

52-80. (canceled)
